(19) United States
(12) Patent Application Publication
(10) Pub. No.: US 2021/0177841 A1
(43) Pub. Date: Jun. 17, 2021

(54) CONJOINT THERAPY WITH GLUTAMINASE INHIBITORS

(71) Applicant: Calithera Biosciences, Inc., South San Francisco, CA (US)

(72) Inventor: Keith Orford, Doylestown, PA (US)

(21) Appl. No.: 16/761,074

(22) PCT Filed: Nov. 1, 2018
(86) PCT No.: PCT/US18/58743

§ 371 Date: May 1, 2020

Related U.S. Application Data
(60) Provisional application No. 62/581,337, filed on Nov. 3, 2017.

(51) Int. Cl. A61K 31/501; C07K 16/28; A61K 45/06; G01N 33/574

(52) U.S. Cl. CPC A61K 31/501; C07K 16/2818; G01N 33/57484; A61K 45/06; C07K 16/2827

(57) ABSTRACT

The invention relates to methods of treating cancer using novel heterocyclic glutaminase inhibitor compounds conjointly with a PD1 or PD-L1 inhibitor.

### RELATED APPLICATION

This application claims the benefit of priority to U.S. Provisional Patent Application No. 62/581,337, filed Nov. 3, 2017, which application is hereby incorporated by reference in its entirety.

### BACKGROUND

Glutamine supports cell survival, growth and proliferation through metabolic and non-metabolic mechanisms. In actively proliferating cells, the metabolism of glutamine to lactate, also referred to as “glutaminolysis” is a major source of energy in the form of NADPH. The first step in glutaminolysis is the deamination of glutamine to form glutamate and ammonia, which is catalyzed by the glutaminase enzyme (GLS). Thus, deamination via glutaminase is a control point for glutamine metabolism.

Ever since Warburg's observation that ascites tumor cells exhibited high rates of glucose consumption and lactate secretion in the presence of oxygen (Warburg, 1956), researchers have been exploring how cancer cells utilize metabolic pathways to be able to continue actively proliferating. Several reports have demonstrated how glutamine metabolism supports macromolecular synthesis necessary for cells to replicate (Curthoys, 1995; DeBardinis, 2008).

Thus, glutaminase has been theorized to be a potential therapeutic target for the treatment of diseases characterized by actively proliferating cells, such as cancer. Recently, glutaminase inhibitor CB-839 has proven to be effective in treating triple-negative breast cancer, thus acting as a proof of concept (Gross, 2014). However, there remains a clinical need for the further utilization of glutaminase inhibitors in the treatment of cancer.

### SUMMARY

The present invention provides a method of treating cancer, such as PD-1 or PD-L1 refractory melanoma, non-small cell lung cancer, or renal cancer in a subject, comprising conjointly administering to the subject:

a PD-1 or a PD-L1 inhibitor, e.g., an anti-PD-1 or an anti-PD-L1 antibody, such as nivolumab, pembrolizumab, pidilizumab, ipilimumab, atezolizumab, avelumab or durvalumab; and

a glutaminase inhibitor, such as a compound of formula (I),

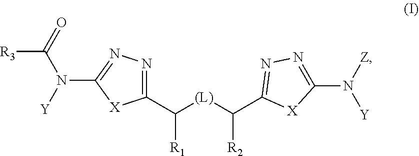

or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or

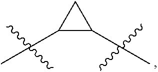 [SMILES: *C1CC1*]

preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂, CH₂CH₂CH₂ or CH₂ may be replaced by hydroxy;
X, independently for each occurrence, represents S, O or CH═CH, preferably S or CH═CH, wherein any hydrogen atom of a CH unit may be replaced by alkyl;
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy;
R₃, independently for each occurrence, represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₆, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H; and
wherein the subject is refractory to treatment with a PD-1 or a PD-L1 inhibitor, such as the anti-PD-1 or the anti-PD-L1 antibody.

In certain embodiments, the present invention provides a pharmaceutical preparation suitable for treating cancer in a human patient refractory to treatment with a PD-1 or a PD-L1 inhibitor in the treatment of cancer, such as melanoma, non-small cell lung cancer (NSCLC), or renal cell carcinoma (RCC), comprising an effective amount of any of the glutaminase inhibitors described herein (e.g., a compound of the invention, such as a compound of formula I), and one or more pharmaceutically acceptable excipients. In certain embodiments, the pharmaceutical preparations may be for use in treating cancer in a subject refractory to treatment with a PD-1 or a PD-L1 inhibitor as described herein.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a tumor sample from a subject having the cancer;

b) measuring the presence, absence, amount, or activity of at least one biomarker listed in Table 2 in the tumor sample; and

c) comparing said presence, absence, amount, or activity of the at least one biomarker listed in Table 2 to a reference standard, e.g., a reference standard representative of a non-responsive refractory tumor,

wherein the presence of the at least one biomarker listed in Table 2 or a significantly increased amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

In certain such embodiments, if the cancer is determined to be likely to be responsive to the conjoint therapy, the method further comprises administering the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a sample from a subject having the cancer, wherein the sample comprises nucleic acid molecules from the tumor;

b) determining the copy number of at least one biomarker listed in Table 2 in the sample; and

c) comparing the copy number to a reference standard, e.g., a reference standard representative of a non-responsive refractory tumor,

wherein an increased copy number of the at least one biomarker listed in Table 2 in the sample relative to the reference standard identifies the cancer as being more likely to be responsive to the conjoint therapy.

In certain such embodiments, if the cancer is identified to be likely to be responsive to the conjoint therapy, the method further comprises administering the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) evaluating expression in the sample of at least one biomarker listed in Table 2, or a combination thereof, compared to a reference standard, e.g., a reference standard representative of a non-responsive refractory tumor,

wherein an increased expression of the at least one biomarker, or a combination thereof, relative to the reference standard, indicates that the conjoint therapy is effective.

In certain such embodiments, if the conjoint therapy is identified to be effective, the method further comprises continuing to administer the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) determining the copy number of at least one biomarker listed in Table 2 in the sample; and

c) comparing the copy number to a reference standard, e.g., a reference standard representative of a non-responsive refractory tumor,

wherein an increased copy number of the at least one biomarker listed in Table 2 in the sample relative to the reference standard indicates that the conjoint therapy is effective.

In certain such embodiments, if the conjoint therapy is identified to be effective, the method further comprises continuing to administer the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a tumor sample from a subject having the cancer;

b) measuring the presence, absence, amount, or activity of at least one biomarker listed in Table 2 in the tumor sample; and

c) comparing said presence, absence, amount, or activity of the at least one biomarker listed in Table 2 to a reference standard, e.g., a reference standard representative of a responsive refractory tumor,

wherein the presence of the at least one biomarker listed in Table 2 or a similar amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

In certain such embodiments, if the cancer is determined to be likely to be responsive to the conjoint therapy, the method further comprises administering the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a sample from a subject having the cancer, wherein the sample comprises nucleic acid molecules from the tumor;

b) determining the copy number of at least one biomarker listed in Table 2 in the sample; and

c) comparing the copy number to a reference standard, e.g., a reference standard representative of a responsive refractory tumor,

wherein a similar copy number of the at least one biomarker listed in Table 2 in the sample relative to the reference standard identifies the cancer as being more likely to be responsive to the conjoint therapy.

In certain such embodiments, if the cancer is determined to be likely to be responsive to the conjoint therapy, the method further comprises administering the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) evaluating expression in the sample of at least one biomarker listed in Table 2, or a combination thereof, compared to a reference standard, e.g., a reference standard representative of a responsive refractory tumor,

wherein a similar expression of the at least one biomarker, or a combination thereof, relative to the reference standard, indicates that the conjoint therapy is effective.

In certain such embodiments, if the conjoint therapy is identified to be effective, the method further comprises continuing to administer the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) determining the copy number of at least one biomarker listed in Table 2 in the sample; and

c) comparing the copy number to a reference standard, e.g., a reference standard representative of a responsive refractory tumor,

wherein a similar copy number of the at least one biomarker listed in Table 2 in the sample relative to the reference standard indicates that the conjoint therapy is effective.

In certain such embodiments, if the conjoint therapy is identified to be effective, the method further comprises continuing to administer the conjoint therapy to the subject.

### BRIEF DESCRIPTION OF THE DRAWINGS

FIG. 1 shows that proliferation of T-cells increased with increasing amounts of glutamine. The T-cells are mouse splenocytes stimulated with anti-CD3/CD28 in presence of varying levels of glutamine.

FIG. 2 shows that CB-839 has a minimal impact on T-cell proliferation.

FIG. 3 shows the CB-839 blockage of tumor cell glutamine consumption and restoration of T-cell division.

FIG. 4 shows the effects of CB-839 on glutamine levels in several in vitro tumor models.

FIG. 5A shows the efficacy of CB-839 and α-PD-L1 inhibitor conjoint treatment in reducing tumor volume in a CT-26 mouse colon cancer model.

FIG. 5B shows the efficacy of CB-839 and α-PD-1 conjoint treatment in reducing tumor volume in a CT-26 mouse colon cancer model.

FIG. 5C shows the efficacy of CB-839 and α-PD-L1 inhibitor conjoint treatment in reducing tumor volume in a B16 mouse melanoma model.

FIG. 5D shows the efficacy of CB-839 and α-PD-L1 inhibitor conjoint treatment in reducing tumor volume in a CT-26 mouse colon cancer model, where one set of mice was pre-treated with α-CD8 antibody.

FIG. 6 shows the cohorts of patients with melanoma, NSCLC, and RCC as refractory prior to treatment with CB-839 and nivolumab.

FIG. 7 shows the progress of the melanoma patients, where partial to complete response were observed.

FIG. 8 shows data on response levels of patients with melanoma, NSCLC, and RCC after treatment with CB-839 and nivolumab.

FIG. 9A shows that elevated levels of biomarkers related to T-cell inflamed signature in pretreatment biopsies associated with clinical benefit. Gene expression was analyzed in biopsies from Melanoma Rescue cohort. To further facilitate the visualization, transcript counts were replaced with colors. Low values are colored in green, high values are colored in red, and average values are colored in black. PR, partial response; CR, complete response; SD, stable disease; and PD, progressive disease.

FIG. 9B shows elevation of biomarkers related to T-cell inflamed signature and effector genes post-treatment with CB-839 and nivolumab in patients with partial response.

FIG. 9C shows elevation of representative biomarkers related to T-cell inflamed signature and effector genes post-treatment with CB-839 and nivolumab in a patient with partial response.

### DETAILED DESCRIPTION

The present invention provides a method of treating cancer, such as melanoma, non-small cell lung cancer, or renal cancer, in a subject refractory to treatment with a PD-1 or a PD-L1 inhibitor, comprising conjointly administering to the subject a PD-1 or a PD-L1 inhibitor, and a glutaminase inhibitor.

In certain embodiments, the glutaminase inhibitor is a compound of formula (I),

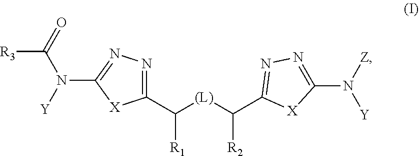

or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or

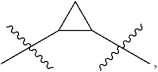 [SMILES: *C1CC1*]

preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂, CH₂CH₂CH₂ or CH₂ may be replaced by hydroxy;
X, independently for each occurrence, represents S, O or CH═CH, preferably S or CH═CH, wherein any hydrogen atom of a CH unit may be replaced by alkyl;
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy;
R₃, independently for each occurrence, represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₆, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H.

In certain embodiments wherein alkyl, hydroxyalkyl, amino, acylamino, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl are substituted, they are substituted with one or more substituents selected from substituted or unsubstituted alkyl, such as perfluoroalkyl (e.g., trifluoromethyl), alkenyl, alkoxy, alkoxyalkyl, aryl, aralkyl, arylalkoxy, aryloxy, aryloxyalkyl, hydroxyl, halo, alkoxy, such as perfluoroalkoxy (e.g., trifluoromethoxy), alkoxyalkoxy, hydroxyalkyl, hydroxyalkylamino, hydroxyalkoxy, amino, aminoalkyl, alkylamino, aminoalkylalkoxy, aminoalkoxy, acylamino, acylaminoalkyl, such as perfluoro acylaminoalkyl (e.g., trifluoromethylacylaminoalkyl), acyloxy, cycloalkyl, cycloalkylalkyl, cycloalkylalkoxy, heterocyclyl, heterocyclylalkyl, heterocyclyloxy, heterocyclylalkoxy, heteroaryl, heteroarylalkyl, heteroarylalkoxy, heteroaryloxy, heteroaryloxyalkyl, heterocyclylaminoalkyl, heterocyclylaminoalkoxy, amido, amidoalkyl, amidine, imine, oxo, carbonyl (such as carboxyl, alkoxycarbonyl, formyl, or acyl, including perfluoroacyl (e.g., C(O)CF₃)), carbonylalkyl (such as carboxyalkyl, alkoxycarbonylalkyl, formylalkyl, or acylalkyl, including perfluoroacylalkyl (e.g., -alkylC(O)CF₃)), carbamate, carbamatealkyl, urea, ureaalkyl, sulfate, sulfonate, sulfamoyl, sulfone, sulfonamide, sulfonamidealkyl, cyano, nitro, azido, sulfhydryl, alkylthio, thiocarbonyl (such as thioester, thioacetate, or thioformate), phosphoryl, phosphate, phosphonate or phosphinate.

In certain embodiments, L represents CH₂SCH₂, CH₂CH₂, CH₂CH₂CH₂, CH₂, CH₂S, SCH₂, or CH₂NHCH₂, wherein any hydrogen atom of a CH₂ unit may be replaced by alkyl or alkoxy, and any hydrogen atom of a CH₂ unit of CH₂CH₂, CH₂CH₂CH₂ or CH₂ may be replaced by hydroxyl. In certain embodiments, L represents CH₂SCH₂, CH₂CH₂, CH₂S or SCH₂. In certain embodiments, L represents CH₂CH₂. In certain embodiments, L is not CH₂SCH₂.

In certain embodiments, Y represents H.

In certain embodiments, X represents S or CH═CH. In certain embodiments, one or both X represents CH═CH. In certain embodiments, each X represents S. In certain embodiments, one X represents S and the other X represents CH═CH.

In certain embodiments, Z represents R₃(CO). In certain embodiments wherein Z is R₃(CO), each occurrence of R₃ is not identical (e.g., the compound of formula I is not symmetrical).

In certain embodiments, R₁ and R₂ each represent H.

In certain embodiments, R₃ represents arylalkyl, heteroarylalkyl, cycloalkyl or heterocycloalkyl. In certain embodiments, R₃ represents C(R₈)(R₉)(R₁₀), wherein R₈ represents aryl, arylalkyl, heteroaryl or heteroaralkyl, such as aryl, arylalkyl or heteroaryl, R₉ represents H, and R₁₀ represents hydroxy, hydroxyalkyl, alkoxy or alkoxyalkyl, such as hydroxy, hydroxyalkyl or alkoxy.

In certain embodiments, L represents CH₂SCH₂, CH₂CH₂, CH₂S or SCH₂, such as CH₂CH₂, CH₂S or SCH₂, Y represents H, X represents S, Z represents R₃(CO), R₁ and R₂ each represent H, and each R₃ represents arylalkyl, heteroarylalkyl, cycloalkyl or heterocycloalkyl. In certain such embodiments, each occurrence of R₃ is identical.

In certain embodiments, L represents CH₂SCH₂, CH₂CH₂, CH₂S or SCH₂, Y represents H, X represents S, Z represents R₃(CO), R₁ and R₂ each represent H, and each R₃ represents C(R₈)(R₉)(R₁₀), wherein R₈ represents aryl, arylalkyl, heteroaryl or heteroaralkyl, such as aryl, arylalkyl or heteroaryl, R₉ represents H, and R₁₀ represents hydroxy, hydroxyalkyl, alkoxy or alkoxyalkyl, such as hydroxy, hydroxyalkyl or alkoxy. In certain such embodiments, each occurrence of R₃ is identical.

In certain embodiments, L represents CH₂CH₂, Y represents H, X represents S or CH═CH, Z represents R₃(CO), R₁ and R₂ each represent H, and each R₃ represents substituted or unsubstituted arylalkyl, heteroarylalkyl, cycloalkyl or heterocycloalkyl. In certain such embodiments, each X represents S. In other embodiments, one or both occurrences of X represents CH═CH, such as one occurrence of X represents S and the other occurrence of X represents CH═CH. In certain embodiments of the foregoing, each occurrence of R₃ is identical. In other embodiments of the foregoing wherein one occurrence of X represents S and the other occurrence of X represents CH═CH, the two occurrences of R₃ are not identical.

In certain embodiments, L represents CH₂CH₂, Y represents H, X represents S, Z represents R₃(CO), R₁ and R₂ each represent H, and each R₃ represents C(R₈)(R₉)(R₁₀), wherein R₈ represents aryl, arylalkyl or heteroaryl, R₉ represents H, and R₁₀ represents hydroxy, hydroxyalkyl or alkoxy. In certain such embodiments, R₈ represents aryl and R₁₀ represents hydroxyalkyl. In certain such embodiments, each occurrence of R₃ is identical.

In certain embodiments wherein L represents CH₂, CH₂CH₂CH₂ or CH₂CH₂, X represents O, and Z represents R₃(CO), both R₃ groups are not alkyl, such as methyl, or C(R₈)(R₉)(R₁₀), wherein R₈, R₉ and R₁₀ are each independently hydrogen or alkyl.

In certain embodiments wherein L represents CH₂CH₂, X represents S, and Z represents R₃(CO), both R₃ groups are not phenyl or heteroaryl, such as 2-furyl.

In certain embodiments wherein L represents CH₂CH₂, X represents 0, and Z represents R₃(CO), both R₃ groups are not N(R₄)(R₅) wherein R₄ is aryl, such as phenyl, and R₅ is H.

In certain embodiments wherein L represents CH₂SCH₂, X represents S, and Z represents R₃(CO), both R₃ groups are not aryl, such as optionally substituted phenyl, aralkyl, such as benzyl, heteroaryl, such as 2-furyl, 2-thienyl or 1,2,4-trizole, substituted or unsubstituted alkyl, such as methyl, chloromethyl, dichloromethyl, n-propyl, n-butyl, t-butyl or hexyl, heterocyclyl, such as pyrimidine-2,4(1H,3H)-dione, or alkoxy, such as methoxy, pentyloxy or ethoxy.

In certain embodiments wherein L represents CH₂SCH₂, X represents S, and Z represents R₃(CO), both R₃ groups are not N(R₄)(R₅) wherein R₄ is aryl, such as substituted or unsubstituted phenyl (e.g., phenyl, 3-tolyl, 4-tolyl, 4-bromophenyl or 4-nitrophenyl), and R₅ is H.

In certain embodiments wherein L represents CH₂CH₂CH₂, X represents S, and Z represents R₃(CO), both R₃ groups are not alkyl, such as methyl, ethyl, or propyl, cycloalkyl, such as cyclohexyl, or C(R₈)(R₉)(R₁₀), wherein any of R₈, R₉ and R₁₀ together with the C to which they are attached, form any of the foregoing.

In further embodiments of the methods of the invention, the glutaminase inhibitor is a compound of formula (Ia),

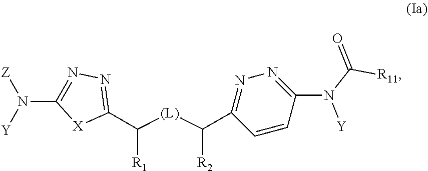

or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or

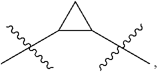 [SMILES: *C1CC1*]

preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂, CH₂CH₂CH₂ or CH₂ may be replaced by hydroxy;
X represents S, O or CH═CH, preferably S or CH═CH, wherein any hydrogen atom of a CH unit may be replaced by alkyl;
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy, preferably H;
R₃ represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₆, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H;
R₁₁ represents substituted or unsubstituted aryl, arylalkyl, aryloxy, aryloxyalkyl, heteroaryl, heteroarylalkyl, heteroaryloxy, or heteroaryloxyalkyl, or C(R₁₂)(R₃)(R₄), N(R₄)(R₁₄) or OR₁₄, wherein any free hydroxyl group may be acylated to form C(O)R₇;
R₁₂ and R₁₃ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇, and wherein both of R₁₂ and R₁₃ are not H; and
R₁₄ represents substituted or unsubstituted aryl, arylalkyl, aryloxy, aryloxyalkyl, heteroaryl, heteroarylalkyl, heteroaryloxy, or heteroaryloxyalkyl; and
a PD-1 or a PD-L1 inhibitor, such as nivolumab, pembrolizumab, pidilizumab, ipilimumab, atezolizumab, avelumab or durvalumab;

wherein the subject is refractory to a PD-1 or a PD-L1 inhibitor, such as a anti-PD-1 or a anti-PD-L1 antibody.

In certain embodiments wherein alkyl, hydroxyalkyl, amino, acylamino, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl are substituted, they are substituted with one or more substituents selected from substituted or unsubstituted alkyl, such as perfluoroalkyl (e.g., trifluoromethyl), alkenyl, alkoxy, alkoxyalkyl, aryl, aralkyl, arylalkoxy, aryloxy, aryloxyalkyl, hydroxyl, halo, alkoxy, such as perfluoroalkoxy (e.g., trifluoromethylalkoxy), alkoxyalkoxy, hydroxyalkyl, hydroxyalkylamino, hydroxyalkoxy, amino, aminoalkyl, alkylamino, aminoalkylalkoxy, aminoalkoxy, acylamino, acylaminoalkyl, such as perfluoro acylaminoalkyl (e.g., trifluoromethylacylaminoalkyl), acyloxy, cycloalkyl, cycloalkylalkyl, cycloalkylalkoxy, heterocyclyl, heterocyclylalkyl, heterocyclyloxy, heterocyclylalkoxy, heteroaryl, heteroarylalkyl, heteroarylalkoxy, heteroaryloxy, heteroaryloxyalkyl, heterocyclylaminoalkyl, heterocyclylaminoalkoxy, amido, amidoalkyl, amidine, imine, oxo, carbonyl (such as carboxyl, alkoxycarbonyl, formyl, or acyl, including perfluoroacyl (e.g., C(O)CF₃)), carbonylalkyl (such as carboxyalkyl, alkoxycarbonylalkyl, formylalkyl, or acylalkyl, including perfluoroacylalkyl (e.g., -alkylC(O)CF₃)), carbamate, carbamatealkyl, urea, ureaalkyl, sulfate, sulfonate, sulfamoyl, sulfone, sulfonamide, sulfonamidealkyl, cyano, nitro, azido, sulfhydryl, alkylthio, thiocarbonyl (such as thioester, thioacetate, or thioformate), phosphoryl, phosphate, phosphonate or phosphinate.

In certain embodiments, R₁ represents substituted or unsubstituted arylalkyl, such as substituted or unsubstituted benzyl.

In certain embodiments, L represents CH₂SCH₂, CH₂CH₂, CH₂CH₂CH₂, CH₂, CH₂S, SCH₂, or CH₂NHCH₂, wherein any hydrogen atom of a CH₂ unit may be replaced by alkyl or alkoxy, and any hydrogen atom of a CH₂ unit of CH₂CH₂, CH₂CH₂CH₂ or CH₂ may be replaced by hydroxyl. In certain embodiments, L represents CH₂SCH₂, CH₂CH₂, CH₂S or SCH₂, preferably CH₂CH₂. In certain embodiments, L is not CH₂SCH₂.

In certain embodiments, each Y represents H. In other embodiments, at least one Y is CH₂O(CO)R₇.

In certain embodiments, X represents S or CH═CH. In certain embodiments, X represents S.

In certain embodiments, R₁ and R₂ each represent H.

In certain embodiments, Z represents R₃(CO). In certain embodiments wherein Z is R₃(CO), R₃ and R₁₁ are not identical (e.g., the compound of formula I is not symmetrical).

In certain embodiments, Z represents R₃(CO) and R₃ represents arylalkyl, heteroarylalkyl, cycloalkyl or heterocycloalkyl. In certain embodiments, Z represents R₃(CO) and R₃ represents C(R₈)(R₉)(R₁₀), wherein R₈ represents aryl, arylalkyl, heteroaryl or heteroaralkyl, such as aryl, arylalkyl or heteroaryl, R₉ represents H, and R₁₀ represents hydroxy, hydroxyalkyl, alkoxy or alkoxyalkyl, such as hydroxy, hydroxyalkyl or alkoxy. In certain embodiments, Z represents R₃(CO) and R₃ represents heteroarylalkyl.

In certain embodiments, L represents CH₂SCH₂, CH₂CH₂, CH₂S or SCH₂, such as CH₂CH₂, Y represents H, X represents S, Z represents R₃(CO), R₁ and R₂ each represent H, R₃ represents arylalkyl, heteroarylalkyl, cycloalkyl or heterocycloalkyl, and R₁₁ represents arylalkyl. In certain such embodiments, R₃ represents heteroarylalkyl.

In certain embodiments, L represents CH₂SCH₂, CH₂CH₂, CH₂S or SCH₂, such as CH₂CH₂, Y represents H, X represents S, Z represents R₃(CO), R₁ and R₂ each represent H, and R₃ represents C(R₈)(R₉)(R₁₀), wherein R₈ represents aryl, arylalkyl, heteroaryl or heteroaralkyl, such as aryl, arylalkyl or heteroaryl, R₉ represents H, and R₁₀ represents hydroxy, hydroxyalkyl, alkoxy or alkoxyalkyl, such as hydroxy, hydroxyalkyl or alkoxy, and R₁₁ represents arylalkyl. In certain such embodiments, R₈ represents heteroaryl.

In certain embodiments, L represents CH₂CH₂, Y represents H, X represents S or CH═CH, such as S, Z represents R₃(CO), R₁ and R₂ each represent H, R₃ represents substituted or unsubstituted arylalkyl, heteroarylalkyl, cycloalkyl or heterocycloalkyl, and R₁₁ represents arylalkyl. In certain such embodiments, R₃ represents heteroarylalkyl.

In certain embodiments, L represents CH₂CH₂, Y represents H, X represents S, Z represents R₃(CO), R₁ and R₂ each represent H, R₃ represents C(R₈)(R₉)(R₁₀), wherein R₈ represents aryl, arylalkyl or heteroaryl, R₉ represents H, and R₁₀ represents hydroxy, hydroxyalkyl or alkoxy, and R₁₁ represents arylalkyl. In certain such embodiments, R₈ represents aryl and R₁₀ represents hydroxyalkyl. In certain other embodiments, R₈ represents heteroaryl. In certain embodiments, the glutaminase inhibitor is selected from any one of the compounds disclosed in Table 3 of PCT Application Publication Number WO 2013/078123, published May 30, 2013, the contents of which are incorporated herein by reference in their entirety.

Preferably, the compound is selected from compound 1, 2, 6, 7, 8, 11, 13, 14, 15, 16, 17, 18, 19, 20, 21, 24, 25, 26, 27, 28, 29, 30, 31, 32, 33, 35, 36, 38, 39, 40, 41, 43, 44, 47, 48, 50, 51, 52, 54, 55, 58, 63, 64, 65, 67, 68, 69, 70, 71, 72, 73, 77, 78, 79, 80, 81, 82, 83, 84, 85, 86, 87, 88, 92, 93, 94, 95, 97, 99, 100, 102, 105, 107, 111, 112, 114, 115, 116, 117, 118, 120, 121, 122, 123, 126, 127, 133, 135, 136, 138, 140, 141, 143, 146, 147, 148, 152, 153, 155, 156, 157, 158, 159, 160, 161, 162, 163, 164, 165, 166, 168, 169, 170, 172, 173, 174, 175, 176, 177, 178, 179, 180, 181, 182, 185, 186, 187, 188, 189, 190, 193, 194, 195, 196, 197, 198, 199, 200, 201, 202, 203, 204, 205, 208, 210, 211, 213, 214, 216, 217, 219, 220, 226, 227, 228, 229, 231, 232, 234, 235, 236, 237, 239, 240, 241, 242, 243, 244, 245, 246, 247, 248, 249, 250, 251, 252, 255, 256, 257, 258, 259, 260, 261, 262, 263, 264, 265, 266, 267, 268, 269, 270, 271, 273, 274, 275, 276, 278, 279, 280, 281, 282, 283, 285, 286, 287, 288, 290, 291, 292, 293, 294, 295, 296, 297, 298, 299, 300, 302, 304, 1038, 306, 307, 308, 309, 310, 311, 313, 314, 315, 316, 317, 318, 319, 320, 321, 322, 323, 324, 325, 327, 329, 332, 333, 334, 335, 336, 337, 338, 339, 340, 341, 342, 343, 344, 345, 346, 527, 347, 348, 349, 350, 351, 352, 353, 354, 355, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 638, 639, 640, 641, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 692, 693, 694, 695, 696, 697, 698, 699, 700, 701, 702, 703, 704, 705, 707, 708, 709, 715, 716, 717, 718, 719, 720, 721, 722, 723, 724, 725, 726, 727, 728, 729, or 730.

In preferred embodiments, the glutaminase inhibitor is Compound 354, also known as CB-839:

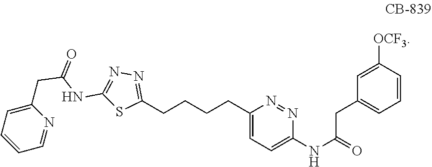 [SMILES: O=C(Cc1cccc(OC(F)(F)F)c1)Nc1ccc(CCCCc2nnc(NC(=O)Cc3ccccn3)s2)nn1]

In certain embodiments, the glutaminase inhibitor used in the methods of the invention is a compound of formula (II),

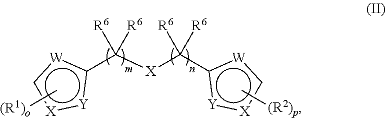

or a pharmaceutically acceptable salt thereof, wherein:
X is a bond, —S—, —S(O)—, —SO₂—, —CH═CH—, or —C(O)—;
each W, Y and Z is independently —S—, —CH═, —O—, —N═, or —NH—, provided that (1) at least one of W, Y and Z is not —CH═ and (2) when one of W is —S— and the Y in the same ring is N, then the Z in the same ring is not —CH═;
each R¹ and R² is independently C₁₋₆ alkylene-R⁴, —N(R³)—R⁴, —N(R³)—C(O) R⁴, —C(O)—N(R³)—R⁴, —N(R³)—C(O)—O—R⁴, —N(R³)—C(O)—N(R³)—R⁴, —O—C(O)—N(R³) R⁴, —N(R³)—C(O)—C₁₋₆ alkylene-C(O)—R⁴, —N(R³)—C(O)—C₁₋₆ alkylene-N(R³)—C(O)—R⁴ or —N(R³^{ₐ})—C(O)—CH₂—N(R³)—C(O)—R⁴;
each R³ is independently hydrogen, C₁₋₆ alkyl or aryl;
each R⁴ is independently C₁₋₆ alkyl, C₁₋₆ alkenyl, aryl, heteroaryl, aralkyl, heteroaralkyl, heterocyclylalkyl, heterocyclyl, cycloalkyl or cycloalkylalkyl, each of which is substituted with 0-3 occurrences of R⁵, or two adjacent R⁵ moieties, taken together with the atoms to which they are attached form a heterocyclyl, heteroaryl, cycloalkyl or aryl;
each R⁵ is independently oxo (═O), C₁₋₆ alkyl, C₁₋₆ haloalkyl, C₁₋₆ alkoxy, cyano, halo, —OH, —SH, —OCF₃, —SO₂—C₁₋₆ alkyl, —NO₂, —N(R⁷)—C(O)— C₁₋₆ alkyl, —N(R⁶)₂, —O—C(O)—C₁₋₆ alkyl, C₃₋₇ cycloalkyl, (C₃₋₇cycloalkyl)alkyl, aryl, aryloxy, C(O)-aryl, heteroaryl, aralkyl, heteroaralkyl, heterocyclylalkyl or heterocyclyl, wherein each aryl, heteroaryl or heterocyclyl is further substituted with 0-3 occurrences of R⁷;
each R⁶ is independently hydrogen, fluoro, OH or C₁₋₆ alkyl;
each R⁷ is independently hydrogen, C₁₋₆ alkyl, —OH, —SH, cyano, halo, —CF₃, —OCF₃, —SO₂—C₁₋₆ alkyl, —NO₂, —N(R⁷)—C(O)—C₁₋₆ alkyl, N(R⁶)₂ or C₁₋₆ alkoxy;
m is 1, 2 or 3;
n is 1, 2 or 3; provided that when X is bond, the sum of m and n is from 3 to 6 and when X is —S—, —S(O)—, —SO₂—, —CH═CH—, or —C(O)—, the sum of m and n is from 2 to 4;
o is 1, 2 or 3; and
p is 1, 2 or 3;
with the proviso that:
(1) when X is —S—, m and n are both 2, each R⁶ is H, then (i) R¹ and R² are not both NHC(O)—R⁴, wherein R⁴ is C₁₋₆ alkyl, monocyclic aryl, monocyclic heteroaryl, monocyclic aralkyl, monocyclic heteroaralkyl and each member of R⁴ is substituted with 0-3 occurrences of R⁵; and (ii) R¹ and R² are not both —NHC(O)O-methyl, —NHC(O)O-ethyl, —NHC(±)-6-pyrimidine-2,4(1H,3H)-dionyl, or —NHC(O)NH— phenyl wherein said phenyl of the —NHC(O)NH-phenyl moiety is optionally substituted with 1 or 2 groups selected from methyl, nitro, and halo;
(2) when X is —S—, m and n are both 1, each R⁶ is H, then (i) R¹ and R² are not both NH-phenyl or —NH-4-methoxy-phenyl;
(3) when X is a bond, the sum of m and n is 3, each R⁶ is H, then R¹ and R² are not both NHC(O)-phenyl;
(4) when X is a bond, m and n are both 2, each R⁶ is H, then R¹ and R² are not both —NHC(O)-furanyl, —NHC(O)-phenyl, —NHC(O)-o-methoxy-phenyl, —NHC(O)—C₁₋₆ alkyl, —NH-benzyl, or —NH-phenyl wherein said phenyl of the NH-phenyl moiety is substituted with 0-3 occurrences of R⁵;
(5) when X is a bond, the sum of m and n is 5, each R⁶ is H, then R¹ and R² are not both NHC(O)—C₁₋₆ alkyl, —NHC(O)-cyclohexyl, or —NH-phenyl wherein said phenyl of the —NH-phenyl moiety is optionally substituted with methyl; and
(6) when X is a bond, m and n are both 3, each R⁶ is H, then R¹ and R² are not both NH-phenyl.

In certain embodiments, W is —S—, each Y is —N═, and each Z is —N═.

In certain embodiments, W is —CH═, each Z is —O—, and each Y is —N═.

In certain embodiments, o is 1 and p is 1.

In certain embodiments, R¹ and R² are each —N(R³)—C(O)—O—R⁴.

In certain embodiments, the compound having the structure of Formula (II) has the structure of Formula (IIa):

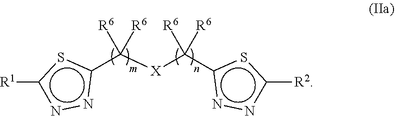

In certain embodiments, R¹ and R² are the same.

In certain embodiments, the compound having the structure of Formula (II) is a compound having the structure of Formula (IIb):

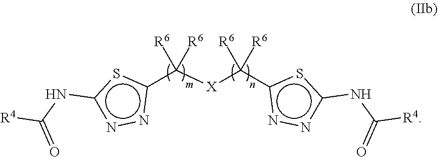

In certain embodiments, the glutaminase inhibitor used in the methods of the invention is a compound of formula (III),

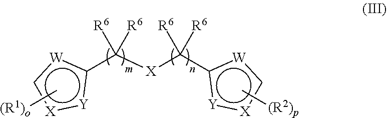

wherein:
X is C₃-C₇ cycloalkylene;
each W, Y and Z is independently —S—, —CH═, —O—, —N═, or —NH—, provided that at least one of W, Y and Z is not —CH═;
each R¹ and R² is independently —NH₂, —N(R³)—C(O)—R⁴, —C(O)—N(R³)—R⁴, —N(R³)—C(O)—O—R⁴, —N(R³)—C(O)—N(R³)—R⁴ or —N(R³)—C(O)—SR⁴;
each R³ is independently hydrogen, C₁₋₆ alkyl or aryl;
each R⁴ is independently C₁₋₆ alkyl, aryl, heteroaryl, aralkyl, heteroaralkyl, cycloalkyl, cycloalkylalkyl, heterocyclylalkyl, or heterocyclyl, each of which is substituted with 0-3 occurrences of R⁵;
each R⁵ is independently C₁₋₆ alkyl, C₁₋₆ alkoxy, —O—C₁₋₆ alkyleneC₁₋₆ alkoxy, C₁₋₆ thioalkoxy, C₁₋₆ haloalkyl, C₃₋₇ cycloalkyl, C₃₋₇ cycloalkylalkyl, aryl, heteroaryl, aralkyl, heteroaralkyl, heterocyclylalkyl, heterocyclyl, cyano, halo, oxo, —OH, —OCF₃, —OCHF₂, —SO₂—C₁₋₆ alkyl, —NO₂, —N(R⁷)—C(O)—C₁₋₆ alkyl, —C(O)N(R⁷)₂, —N(R⁷)S(O)₁₋₂—C₁₋₆ alkyl, —S(O)₂N(R⁷)₂, —N(R⁷)₂, —C₁₋₆ alkylene-N(R⁷)₂, wherein said alkyl, C₁₋₆ alkoxy, —O—C₁₋₆ alkyleneC₁₋₆alkoxy, C₁₋₆ thioalkoxy, C₁₋₆ haloalkyl, C₃₋₇ cycloalkyl, C₃₋₇ cycloalkylalkyl, aryl, heteroaryl, aralkyl, heteroaralkyl, heterocyclylalkyl, heterocyclyl, —SO₂—C₁₋₆alkyl, —NO₂, —N(R⁷)—C(O)—C₁₋₆ alkyl, —C(O)N(R⁷)₂, —N(R⁷)S(O)₁₋₂—C₁₋₆alkyl, —S(O)₂N(R⁷)₂, —N(R⁷)₂, or —C₁₋₆ alkylene-N(R⁷)₂ is optionally substituted with 0-3 occurrences of R⁸; or two adjacent R⁵ moieties, taken together with the atoms to which they are attached form a cycloalkyl or heterocyclyl;
each R⁶ is independently hydrogen, fluoro, C₁₋₆ alkyl, —OH, —NH₂, —NH(CH₃), —N(CH₃)₂, or C₁₋₆ alkoxy;
each R⁷ is independently hydrogen or C₁₋₆ alkyl;
each R⁸ is independently halo, C₁₋₆ alkyl, C₁₋₆ haloalkyl, —OH, —N(R⁷)₂, or C₁₋₆ alkoxy, —O—C₁₋₆ alkyleneC₁₋₆ alkoxy, CN, NO₂, —N(R⁷)—C(O)—C₁₋₆ alkyl, —C(O)N(R⁷)₂, —N(R⁷)S(O)₁₋₂C₁₋₆ alkyl, or —S(O)₂N(R⁷)₂;
m is 0, 1, or 2;
n is 0, 1, or 2;
o is 1, 2 or 3; and
p is 1, 2 or 3; provided that (1) when X is unsubstituted cyclopropyl, R¹ and R² are not both NH-phenyl; and (2) X is other than substituted cyclobutyl or substituted cyclopentyl;

In certain embodiments, W is —S—, each Y is —N═, and each Z is —N═.

In certain embodiments, o is 1 and p is 1.

In certain embodiments, m is 0 and n is 0. Alternatively, m and n can each be 1.

In certain embodiments, R¹ and R² are different. Alternatively, R¹ and R² can be the same.

In certain embodiments, R¹ and R² are each —N(R³)—C(O)—O—R⁴, wherein each R³ is hydrogen and each R⁴ is aralkyl or heteroaralkyl, each of which is substituted with 0-3 occurrences of R⁵.

In certain embodiments, the compound having the structure of Formula (III) is a compound having the structure of Formula (IIIa):

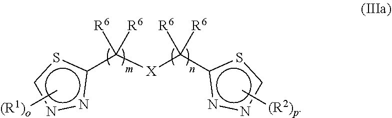

In certain embodiments, the compound having the structure of Formula (III) is a compound having the structure of Formula (IIIb):

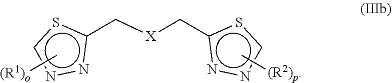

In certain embodiments, the compound having the structure of Formula (III) has the structure of formula (IIIc):

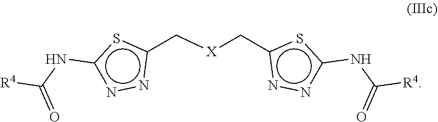

In certain embodiments, the compound of formula (III) is a compound of formula (IV):

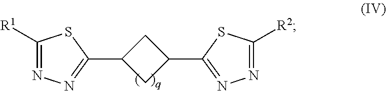

wherein q is 0, 1, 2, 3, or 4.

In certain embodiments, the compound of formula (III) has the structure of formula (IVa):

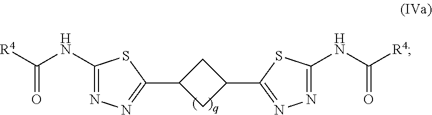

wherein q is 0, 1, 2, 3, or 4.

In certain embodiments, the compound of formula (III) has the structure of formula (IVb).

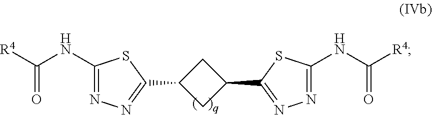

wherein q is 0, 1, 2, 3, or 4.

In certain embodiments, the compound of formula (III) has the structure of formula (IVc).

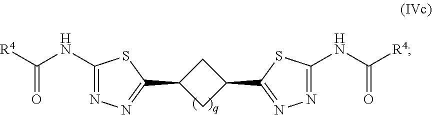

wherein q is 0, 1, 2, 3, or 4.

Compounds of any of Formulae (I) to (IV) are alternatively referred to herein as “glutaminase inhibitors.”

PD-1 and PD-L1 inhibitors are a class of chemotherapeutics. PD-1 and PD-L1 inhibitors have been used in treating a number of cancers, such as bladder cancer, breast cancer, esophageal cancer, gastric cancer, head & neck cancer, Kaposi's sarcoma, lung cancer (including non-small cell lung cancer and small cell lung cancer), melanoma, ovarian cancer, pancreatic cancer, penile cancer, prostate cancer, testicular germcell cancer, thymoma and thymic carcinoma. Representative PD-1 and PD-L1 inhibitors include nivolumab, pembrolizumab, pidilizumab, ipilimumab, atezolizumab, avelumab or durvalumab.

As used herein, the term “refractory” describes a subject whose disease (e.g., tumor) is unresponsive to a PD-1 or PD-L1 inhibitor. Refractory subjects can have a lesser response than the treatment's efficacy in typical, responsive patients, a response that diminishes or terminates after an initial period of responsiveness to the treatment, or no response to the treatment (e.g., the tumor continues to grow). A “response” to a disclosed method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others. In the treatment of cancer, a response typically indicates a reduced rate of growth for a tumor, a cessation of tumor growth, or a shrinkage of a tumor. Similarly, a response may indicate a lack of new tumors (metastases). A refractory subject, on the other hand, may experience tumor growth or the appearance of additional tumors (metastases) despite receiving the therapeutic treatment. Subjects that are refractory to a treatment may have responded initially but then became resistant to the treatment overtime. Other subjects never significantly respond to the treatment.

In certain embodiments, a subject may be refractory to any PD-1 or PD-L1 inhibitor. Subjects as described herein have already been dosed with one or more PD-1 or PD-L1 inhibitors, or even the PD-1 or PD-L1 inhibitor administered conjointly with the glutaminase inhibitor. Representative PD-1 and PD-L1 inhibitors include anti-PD-1 and anti-PD-L1 antibodies, such as nivolumab, pembrolizumab, pidilizumab, ipilimumab, atezolizumab, avelumab and durvalumab.

In further embodiments of the invention, the cancer is refractory to treatment with a PD-1 and PD-L1 inhibitor and selected from bladder cancer, breast cancer, esophageal cancer, gastric cancer, head & neck cancer, Kaposi's sarcoma, lung cancer (including non-small cell lung cancer and small cell lung cancer), melanoma, ovarian cancer, pancreatic cancer, penile cancer, prostate cancer, testicular germcell cancer, thymoma and thymic carcinoma.

In certain embodiments, compounds of the invention may be prodrugs of the compounds of formula (I) to (IV), e.g., wherein a hydroxyl in the parent compound is presented as an ester or a carbonate, or carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, compounds of the invention may be racemic. In certain embodiments, compounds of the invention may be enriched in one enantiomer. For example, a compound of the invention may have greater than about 30% ee, about 40% ee, about 50% ee, about 60% ee, about 70% ee, about 80% ee, about 90% ee, or even about 95% or greater ee. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of formula I or Ia). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of formula (I) to (IV)). A diastereomerically enriched mixture may comprise, for example, at least about 60 mol percent of one diastereomer, or more preferably at least about 75, about 90, about 95, or even about 99 mol percent.

In certain embodiments, the present invention provides a pharmaceutical preparation suitable for use in a human patient, comprising any of the compounds shown above (e.g., a compound of the invention, such as a compound of formula I or Ia), and one or more pharmaceutically acceptable excipients. In certain embodiments, the pharmaceutical preparations may be for use in treating a condition or disease as described herein.

Any of the disclosed compounds may be used in the manufacture of medicaments for the treatment of any diseases or conditions disclosed herein.

### Uses of PD-1 and PD-L1 Inhibitors

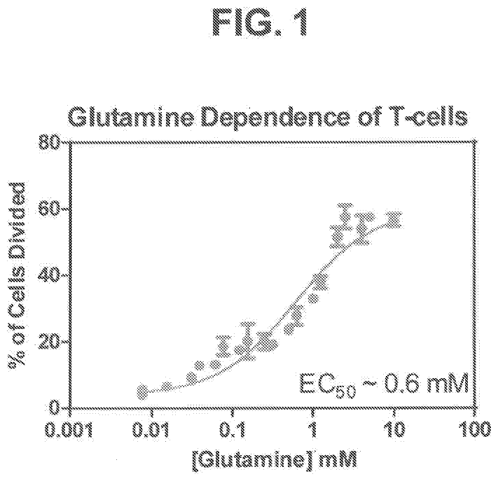

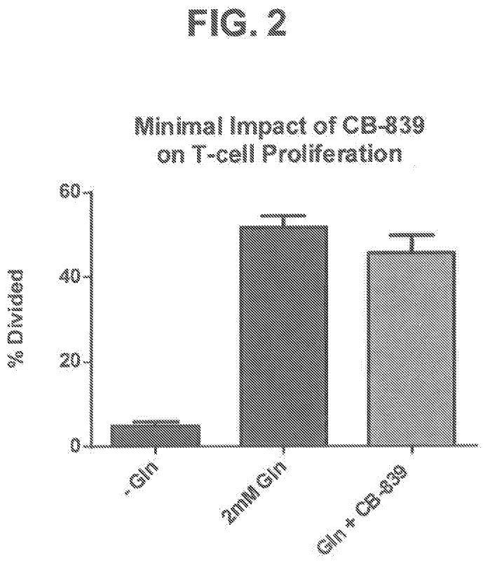

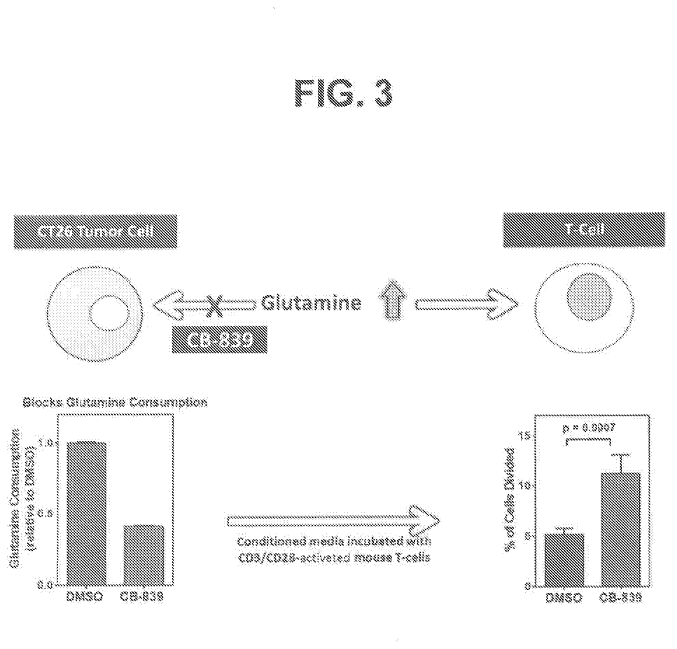

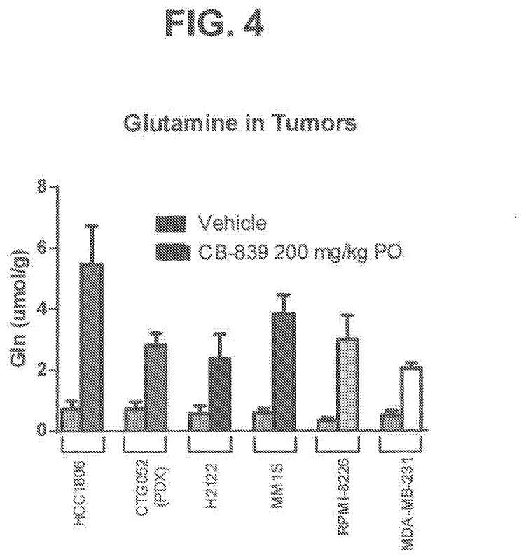

T-cells utilize both glucose and glutamine for proliferation. See, FIG. 1. Their metabolism is suppressed in tumor microenvironments because the tumor cells consume a significant amount of available glucose and glutamine. PD-1 expression and ligation acts to reduce the glucose and glutamine uptake by the tumor cells. The compounds disclosed herein, such as CB-839, block glutamine consumption by tumor cells. However, such compounds do not block glutamine consumption by T-cells or inhibit T-cell proliferation. See, FIG. 2. Thus, the disclosed compounds inhibit tumor cell proliferation while allowing normal metabolic function of T-cells. See, FIG. 3. CB-839 affects glutamine levels in many tumor cell lines as shown in FIG. 4.

In certain embodiments, the PD-1 or PD-L1 inhibitor can be selected from nivolumab, pembrolizumab, pidilizumab, ipilimumab, atezolizumab, avelumab and durvalumab.

In certain embodiments, the disclosed methods may further comprise administering one or more other chemotherapeutic agent(s). Chemotherapeutic agents that may be conjointly administered include: ABT-263, afatinib dimaleate, axitinib, aminoglutethimide, amsacrine, anastrozole, asparaginase, AZD5363, Bacillus Calmette-Guerin vaccine (bcg), bicalutamide, bleomycin, bortezomib, buserelin, busulfan, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, procarbazine, raltitrexed, ramucirumab, rituximab, romidepsin, rucaparib, selumetinib, sirolimus, sorafenib, streptozocin, sunitinib, suramin, talazoparib, tamoxifen, temozolomide, temsirolimus, teniposide, testosterone, thalidomide, thioguanine, thiotepa, titanocene dichloride, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered: ABT-263, dexamethasone, 5-fluorouracil, PF-04691502, romidepsin, and vorinostat (SAHA).

Many combination therapies have been developed for the treatment of cancer. In certain embodiments, methods of the invention may include conjointly administration with a therapy included in Table 1.

TABLE 1
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC | Cyclophosphamide, Doxorubicin |
| (Neuroblastoma) | |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, Cyclophos- |
| | phamide, Vincristine, Procarbazine, |
| | Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide,Epirubicin, Fluorouracil |
| CEPP(B) | Cyclophosphamide, Etoposide, Prednisone, with or |
| | without/Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, |
| | Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, |
| | Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, |
| | Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, |
| | Prednisone |
| CP(Chronic | Chlorambucil, Prednisone |
| lymphocytic | |
| leukemia) | |
| CP | Cyclophosphamide, Cisplatin |
| (Ovarian Cancer) | |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, |
| | Prednisone |
| MAID | Mesna, Doxorubicin, Ifosfamide, Dacarbazine |
| m-BACOD | Bleomycin, Doxorubicin, Cyclophosphamide, |
| | Vincristine, Dexamethasone, Methotrexate, |
| | Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, |
| | Prednisone |
| MOPP/ABV | Mechlorethamine, Vincristine, Procarbazine, |
| | Prednisone, Doxorubicin, Bleomycin, Vinblastine |
| MP (multiple | Melphalan, Prednisone |
| myeloma) | |
| MP (prostate | Mitoxantrone, Prednisone |
| cancer) | |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast | Mitomycin, Vinblastine |
| cancer) | |
| MV (acute | Mitoxantrone, Etoposide |
| myelocytic | |
| leukemia) | |
| M-VAC | Vinblastine, Doxorubicin, Cisplatin |
| Methotrexate | |
| MVP | Vinblastine, Cisplatin |
| Mitomycin | |
| MVPP | Mechlorethamine, Vinblastine, Procarbazine, |
| | Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/ | Prednisone, Doxorubicin, Cyclophosphamide, |
| cytaBOM | Etoposide, Cytarabine, Bleomycin, Vincristine, |
| | Methotrexate, Leucovorin, Cotrimoxazole |
| PRoMACE/ | Prednisone, Doxorubicin, Cyclophosphamide, |
| MOPP | Etoposide, Mechlorethamine, Vincristine, Pro- |
| | carbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophos- |
| | phamide, Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, |
| | Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, |
| | Procarbazine, Hydroxyurea, Cisplatin, Cytarabine, |
| | Dacarbazine |

Examples of combination therapies suitable for use in the methods of the invention include cisplatin and fluorouracil; and ifosfamide, mesna, and cisplatin.

In certain embodiments, the conjoint therapies of the invention further comprise conjoint administration with other types of chemotherapeutic agents, such as immuno-oncology agents. Cancer cells often have specific cell surface antigens that can be recognized by the immune system. Thus, immuno-oncology agents, such as monoclonal antibodies, can selectively bind to cancer cell antigens and effect cell death. Other immuno-oncology agents can suppress tumor-mediated inhibition of the native immune response or otherwise activate the immune response and thus facilitate recognition of the tumor by the immune system. Exemplary immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, and tremelimumab. In some embodiments, the immuno-oncology agent is an anti-CTLA-4 agent, including, but not limited to, ipilimumab and tremelimumab. Thus, in some embodiments, the methods of the invention further comprise conjoint administration of one or more immuno-oncology agents, such as the agents mentioned above.

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, with cryotherapy, or with any combination of these.

In certain embodiments, the present invention provides a kit comprising: a) one or more single dosage forms of a glutaminase inhibitor; b) one or more single dosage forms of a PD-1 or a PD-L1 inhibitor, such as nivolumab, pembrolizumab, pidilizumab, ipilimumab, atezolizumab, avelumab or durvalumab; and c) instructions for the administration of the glutaminase inhibitor and the PD-1 or the PD-L1 inhibitor, for the treatment of cancer, such as melanoma, non-small cell lung cancer or renal cancer, in a subject refractory to treatment with a PD-1 or PD-L1 inhibitor.

The present invention provides a kit comprising:
a) a pharmaceutical formulation (e.g., one or more single dosage forms) comprising a glutaminase inhibitor and a PD-1 or a PD-L1 inhibitor; and
b) instructions for the administration of the pharmaceutical formulation, e.g., for treating cancer, such as melanoma, non-small cell lung cancer, or renal cancer in a subject refractory to treatment with a PD-1 or PD-L1 inhibitor.

In certain embodiments, the kit further comprises instructions for the administration of the pharmaceutical formulation comprising a glutaminase inhibitor conjointly with a PD-1 or a PD-L1 inhibitor, as mentioned above. In certain embodiments, the kit further comprises a second pharmaceutical formulation (e.g., as one or more single dosage forms) comprising a chemotherapeutic agent as mentioned above.

### Biomarkers

Disclosed herein are certain biomarkers that correlate with clinical outcome in cancer, such as melanoma, non-small cell lung cancer or renal cancer, in a subject refractory to treatment with a PD-1 or PD-L1 inhibitor. In addition, provided herein are methods for stratifying patients who are predicted to respond to a glutaminase inhibitor and PD-1 or PD-L1 inhibitor conjoint therapy based upon a determination and analysis of biomarkers described herein according to amount (e.g., copy number or level of expression) and/or activity, relative to a control. In addition, such analyses can be used in order to provide useful therapeutic regimens (e.g., based on predictions of clinical response, subject survival or relapse, timing of adjuvant or neoadjuvant treatment, etc.).

In certain embodiments, the present invention provides a pharmaceutical preparation suitable for treating cancer, such as melanoma, non-small cell lung cancer (NSCLC), or renal cell carcinoma (RCC), in a human patient refractory to treatment with a PD-1 or PD-L1 inhibitor comprising an effective amount of any of the glutaminase inhibitors described herein (e.g., a compound of the invention, such as a compound of formula I), and one or more pharmaceutically acceptable excipients. In certain embodiments, the pharmaceutical preparations may be for use in treating a condition or disease as described herein.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a tumor sample from a subject having the cancer;

b) measuring the presence, absence, amount, or activity of at least one biomarker listed in Table 2 in the tumor sample; and

c) comparing said presence, absence, amount, or activity of the at least one biomarker listed in Table 2 to a reference standard, e.g., a reference standard representative of a non-responsive refractory tumor,

wherein the presence of the at least one biomarker listed in Table 2 or a significantly increased amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

In certain such embodiments, if the cancer is determined to be likely to be responsive to the conjoint therapy, the method further comprises administering the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a sample from a subject having the cancer, wherein the sample comprises nucleic acid molecules from the tumor;

b) determining the copy number of at least one biomarker listed in Table 2 in the sample; and

c) comparing the copy number to a reference standard, e.g., a reference standard representative of a non-responsive refractory tumor,

wherein an increased copy number of the at least one biomarker listed in Table 2 in the sample relative to the reference standard identifies the cancer as being more likely to be responsive to the conjoint therapy.

In certain such embodiments, if the cancer is identified to be likely to be responsive to the conjoint therapy, the method further comprises administering the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) evaluating expression in the sample of at least one biomarker listed in Table 2, or a combination thereof, compared to a reference standard, e.g., a reference standard representative of a non-responsive refractory tumor, wherein an increased expression of the at least one biomarker, or a combination thereof, relative to the reference standard, indicates that the conjoint therapy is effective.

In certain such embodiments, if the conjoint therapy is identified to be effective, the method further comprises continuing to administer the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) determining the copy number of at least one biomarker listed in Table 2 in the sample; and

c) comparing the copy number to a reference standard, e.g., a reference standard representative of a non-responsive refractory tumor,

wherein an increased copy number of the at least one biomarker listed in Table 2 in the sample relative to the reference standard indicates that the conjoint therapy is effective.

In certain such embodiments, if the conjoint therapy is identified to be effective, the method further comprises continuing to administer the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a tumor sample from a subject having the cancer;

b) measuring the presence, absence, amount, or activity of at least one biomarker listed in Table 2 in the tumor sample; and

c) comparing said presence, absence, amount, or activity of the at least one biomarker listed in Table 2 to a reference standard, e.g., a reference standard representative of a responsive refractory tumor,

wherein the presence of the at least one biomarker listed in Table 2 or a similar amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

In certain such embodiments, if the cancer is determined to be likely to be responsive to the conjoint therapy, the method further comprises administering the conjoint therapy to the subject.

In certain embodiments, provided herein are methods for identifying the likelihood of a cancer, such as melanoma, non-small cell lung cancer (NSCLC), or renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:

a) obtaining or providing a sample from a subject having the cancer, wherein the sample comprises nucleic acid molecules from the tumor;

b) determining the copy number of at least one biomarker listed in Table 2 in the sample; and

c) comparing the copy number to a reference standard, e.g., a reference standard representative of a responsive refractory tumor,

wherein a similar copy number of the at least one biomarker listed in Table 2 in the sample relative to the reference standard identifies the cancer as being more likely to be responsive to the conjoint therapy.

In certain such embodiments, if the cancer is determined to be likely to be responsive to the conjoint therapy, the method further comprises administering the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) evaluating expression in the sample of at least one biomarker listed in Table 2, or a combination thereof, compared to a reference standard, e.g., a reference standard representative of a responsive refractory tumor,

wherein a similar expression of the at least one biomarker, or a combination thereof, relative to the reference standard, indicates that the conjoint therapy is effective.

In certain such embodiments, if the conjoint therapy is identified to be effective, the method further comprises continuing to administer the conjoint therapy to the subject.

In certain embodiments, provided herein are methods of monitoring an effect of conjoint therapy with a glutaminase inhibitor and a PD-1 or a PD-L1 inhibitor to treat a cancer, such as melanoma, non-small cell lung cancer (NSCLC), renal cell carcinoma (RCC), in a subject refractory to treatment with a PD-1 or PD-L1 inhibitor, comprising:

(a) obtaining a tumor sample from the subject; and

(b) determining the copy number of at least one biomarker listed in Table 2 in the sample; and

c) comparing the copy number to a reference standard, e.g., a reference standard representative of a responsive refractory tumor,

wherein a similar copy number of the at least one biomarker listed in Table 2 in the sample relative to the reference standard indicates that the conjoint therapy is effective.

In certain such embodiments, if the conjoint therapy is identified to be effective, the method further comprises continuing to administer the conjoint therapy to the subject.

TABLE 2
T-Cell Inflamed
Gene Signature
CD8A
CD274
PDCD1LG2
LAG3
TIGIT
IDO1
CMKLR1
CD27
CXCL9
CXCR6
CCL
CD276
STAT1
PSMB10
HLA-E
HLA-DQ1A
PRF1
GZMA
GZMB

Disclosed herein are methods of assessing the efficacy of a glutaminase inhibitor and PD-1 or PD-L1 inhibitor conjoint therapy for treating cancer, such as melanoma, non-small cell lung cancer (NSCLC) and renal cell cancer (RCC), in a subject, comprising: a) detecting in a first subject sample and maintained in the presence of the therapy the presence, absence, amount, or activity of at least one biomarker listed in Table 2; b) detecting the presence, absence, amount, or activity of the at least one biomarker listed in Table 2 in a second subject sample and maintained in the absence of the glutaminase inhibitor and PD1 or PD-L1 inhibitor; and c) comparing the presence, absence, amount, or activity of the at least one biomarker listed in Table 2 from steps a) and b), wherein a presence or a significantly increased amount or activity of the at least one biomarker listed in Table 2 in the first subject sample relative to at least one subsequent subject sample, indicates that the glutaminase inhibitor and PD1 or PD-L1 inhibitor conjoint therapy treats the cancer in the subject, where the subject is refractory to treatment with a PD-1 or PD-L1 inhibitor. In some embodiments, an absence or an insignificantly increased amount or activity of the at least one biomarker listed in Table 2 in the first subject sample relative to at least one subsequent subject sample, indicates that the dose of the glutaminase inhibitor and PD1 or PD-L1 inhibitor conjoint therapy should be increased.

Further embodiments include methods of assessing the efficacy of glutaminase inhibitor and PD1 or PD-L1 inhibitor conjoint therapy for treating a cancer, such asmelanoma, non-small cell lung cancer (NSCLC) and renal cell cancer (RCC), in a subject, comprising: a) detecting in a subject sample at a first point in time the presence, absence, amount, or activity of at least one biomarker listed in Table 2; b) repeating step a) during at least one subsequent point in time after administration of the glutaminase inhibitor and PD1 or PD-L1 inhibitor conjoint therapy; and c) comparing the presence, absence, amount, or activity detected in steps a) and b), wherein a presence or a significantly increased amount or activity of the at least one biomarker listed in Table 2 in the first subject sample relative to at least one subsequent subject sample, indicates that the glutaminase inhibitor and PD1 or PD-L1 inhibitor conjoint combination therapy treats the cancer in the subject, where the subject is refractory to treatment with a PD-1 or PD-L1 inhibitor. In some embodiments, between the first point in time and the subsequent point in time, the subject has undergone treatment, completed treatment, and/or is in remission for cancer. In other embodiments, the first and/or at least one subsequent sample is selected from ex vivo or in vivo samples. In other embodiments, the first and/or at least one subsequent sample is obtained from an animal model of a cancer, such as melanoma, non-small cell lung cancer (NSCLC) and renal cell cancer (RCC). In some embodiments, the first and/or at least one subsequent sample is a portion of a single sample or pooled samples obtained from the subject.

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. Co alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

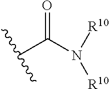

wherein each R¹⁰ independently represents a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

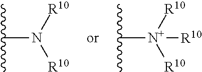

wherein each R¹⁰ independently represents a hydrogen or a hydrocarbyl group, or two R¹⁰ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

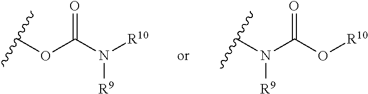

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In some embodiments, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

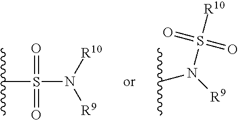

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

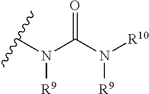

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, biomarker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “biomarker” refers to a measurable entity that has been determined to be predictive of glutaminase inhibitor therapy effects on a cancer. Biomarkers can include, without limitation, nucleic acids (e.g., genomic nucleic acids and/or transcribed nucleic acids) and proteins, including those shown in Table 2, the Examples, and the Figures.

A reference standard expression product level may be determined from any suitable source, including but not limited to a sample of a refractory tumor that is responsive to conjoint therapy with a glutaminase inhibitor and a PD-1 or PD-L1 inhibitor, a sample of a refractory tumor that in not responsive to conjoint therapy with a glutaminase inhibitor and a PD-1 or PD-L1 inhibitor, or a previously determined expression product level range within a test sample from a group of patients, or a set of patients with a certain outcome (for example, reducing tumor burden over 6 months, one year, two years or more).

A reference standard may correlate with one or more values from a subject that is responding to a given treatment. Conversely, a reference standard may correlate with one or more values from a subject that is not responding to a given treatment.

It will be understood by those of skill in the art that such reference standard expression product levels can be used in combination in the methods of the present invention. For example, a reference standard can be a value obtained from a sample from a subject that is responsive to treatments disclosed herein. Another reference standard can be obtained from a sample from a subject that is non-responsive to treatments disclosed herein. In some embodiments, the reference standard may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level.

In some embodiments, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In some embodiments, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-off point in comparing the level of expression product in the test sample to the control.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the reference expression activity or level of the biomarker, e.g., an average expression level of the biomarker in several reference samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the reference expression level of the biomarker, e.g., the average expression level of the biomarker in several reference samples.

The term “similar expression” of a biomarker refers to an expression level in a test sample that is within the standard error of the assay employed to assess expression, and is preferably no more than 10%, and more preferably no more than 1%, 2%, 3%, 4%, 5%, 6%, 7%, 8%, 9%, or 10% different than the reference expression level of the biomarker, e.g., the average expression level of the biomarker in several reference samples.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to glutaminase inhibitor therapy. Such predictive use of the biomarker may be confirmed by, e.g., (1) increased or decreased copy number (e.g., by FISH, FISH plus SKY, single-molecule sequencing, e.g., as described in the art at least at Augustin et al. (2001) J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, or plasma, from a subject, e.g., a human, afflicted with cancer; (3) its absolute or relatively modulated presence or absence in clinical subset of patients with cancer (e.g., those responding to a particular therapy or those developing resistance thereto).

The term “response to anti-cancer therapy” relates to any response of the hyperproliferative disorder (e.g., cancer) to an anti-cancer agent, preferably to a change in tumor mass and/or volume after initiation of neoadjuvant or adjuvant chemotherapy.

Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “sample” used for detecting or determining the presence or level of at least one biomarker is typically tissue (e.g., biopsy), whole blood, plasma, or serum. In certain instances, the method of the present invention further comprises obtaining the sample from the individual prior to detecting or determining the presence or level of at least one marker in the sample.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

In some embodiments of the present invention, the change of biomarker amount and/or activity measurement(s) from the reference standard is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement. In some embodiments of the present invention, the change of biomarker amount and/or activity measurement(s) from the reference standard is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold or greater. In some embodiments, the fold change is less than about 1, less than about 5, less than about 10, less than about 20, less than about 30, less than about 40, or less than about 50. In other embodiments, the fold change in biomarker amount and/or activity measurement(s) compared to the reference standard is more than about 1, more than about 5, more than about 10, more than about 20, more than about 30, more than about 40, or more than about 50.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In some embodiments, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well known to those of skill in the art. The presence or absence of chromosomal gain or loss can be evaluated simply by a determination of copy number of the regions or markers identified herein.

In some embodiments, a biological sample is tested for the presence of copy number changes in genomic loci containing the genomic marker. In some embodiments, a biological sample is tested for the presence of copy number changes in genomic loci containing the genomic marker. The absence of at least one biomarker listed in Table 2 is predictive of poorer outcome of therapy. A copy number of at least 3, 4, 5, 6, 7, 8, 9, or 10 of at least one biomarker listed in Table 2 is predictive of likely response to therapy.

Biomarker expression may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In some embodiments, detecting or determining expression levels of a biomarker and functionally similar homologs thereof, including a fragment or genetic alteration thereof (e.g., in regulatory or promoter regions thereof) comprises detecting or determining RNA levels for the marker of interest. In some embodiments, one or more cells from the subject to be tested are obtained and RNA is isolated from the cells. In some embodiments, a sample of breast tissue cells is obtained from the subject.

b. Methods for Detection of Biomarker Gene Expression

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In some embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to glutaminase inhibitor therapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

Some embodiments relate to screening assays, including non-cell based assays. In some embodiments, the assays provide a method for identifying whether a cancer is likely to respond to anti-cancer therapy (e.g., glutaminase inhibitor therapy) and/or whether an agent can inhibit the growth of or kill a cancer cell that is unlikely to respond to anti-cancer therapy (e.g., glutaminase inhibitor therapy).

In some embodiments, the invention relates to assays for screening test agents which bind to, or modulate the biological activity of, at least one biomarker listed in Table 2. In some embodiments, a method for identifying such an agent entails determining the ability of the agent to modulate, e.g. upregulate, the at least one biomarker listed in Table 2.

In some embodiments, an assay is a cell-free or cell-based assay, comprising contacting at least one biomarker listed in Table 2, with a test agent, and determining the ability of the test agent to modulate (e.g. upregulate) the enzymatic activity of the biomarker, such as by measuring direct binding of substrates or by measuring indirect parameters as described below.

In some embodiments, an assay is a cell-free or cell-based assay, comprising contacting at least one biomarker listed in Table 2, with a test agent, and determining the ability of the test agent to modulate (e.g. upregulate) the ability of the biomarker to regulate translation of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies of the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In some embodiments, determining the ability of the agent to modulate the interaction between the biomarker and its natural binding partner can be accomplished by determining the ability of the test agent to modulate the activity of a polypeptide or other product that functions downstream or upstream of its position within the gene.

The present invention also pertains to the field of predictive medicine in which diagnostic assays, prognostic assays, and monitoring clinical trials are used for prognostic (predictive) purposes to thereby treat an individual prophylactically. Accordingly, some embodiments relate to diagnostic assays for determining the amount and/or activity level of a biomarker listed in Table 2 in the context of a biological sample (e.g., blood, serum, cells, or tissue) to thereby determine whether an individual afflicted with a cancer is likely to respond to glutaminase inhibitor therapy, whether in an original or recurrent cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers listed in Table 2.

Some embodiments relate to monitoring the influence of agents (e.g., drugs, compounds, and small nucleic acid-based molecules) on the expression or activity of a biomarker listed in Table 2. These and other agents are described in further detail in the following sections.

An exemplary method for detecting the amount or activity of a biomarker listed in Table 2, and thus useful for classifying whether a sample is likely or unlikely to respond to glutaminase inhibitor therapy involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of formula I or Ia) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

### Example 1: Synthesis of CB-839: 2-phenyl-N-(6-(4-(5-(2-(pyridin-2-yl)acetamido)-1,3,4-thiadiazol-2-yl)butyl)pyridazin-3-yl)acetamide

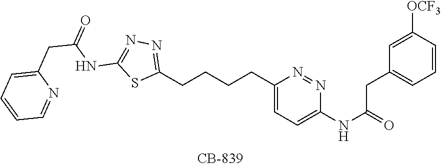 [SMILES: O=C(Cc1cccc(OC(F)(F)F)c1)Nc1ccc(CCCCc2nnc(NC(=O)Cc3ccccn3)s2)nn1]

The synthesis of CB-839 is as described for compound 354 in WO 2014/078645, incorporated herein by reference in its entirety.

### Example 2: Combination of CB-839 and α-PD-L1 Reduces Tumor Volume in Mouse Models

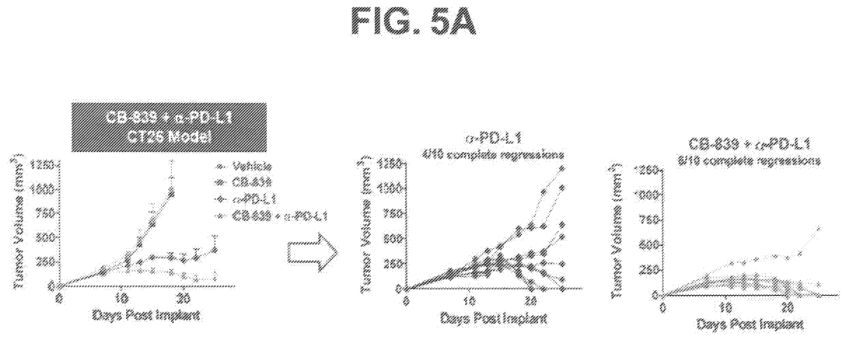

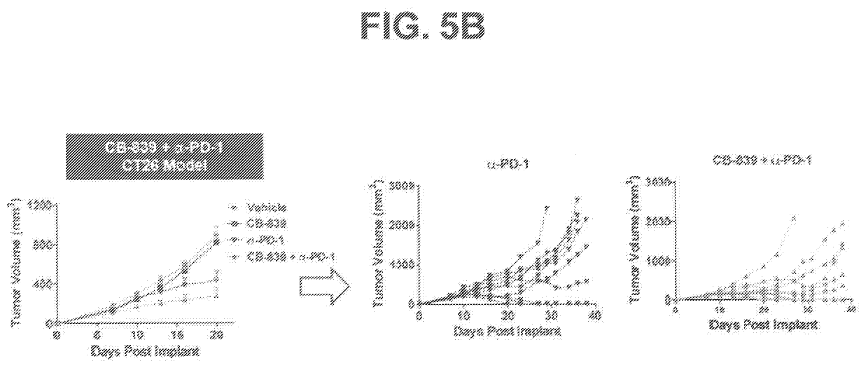

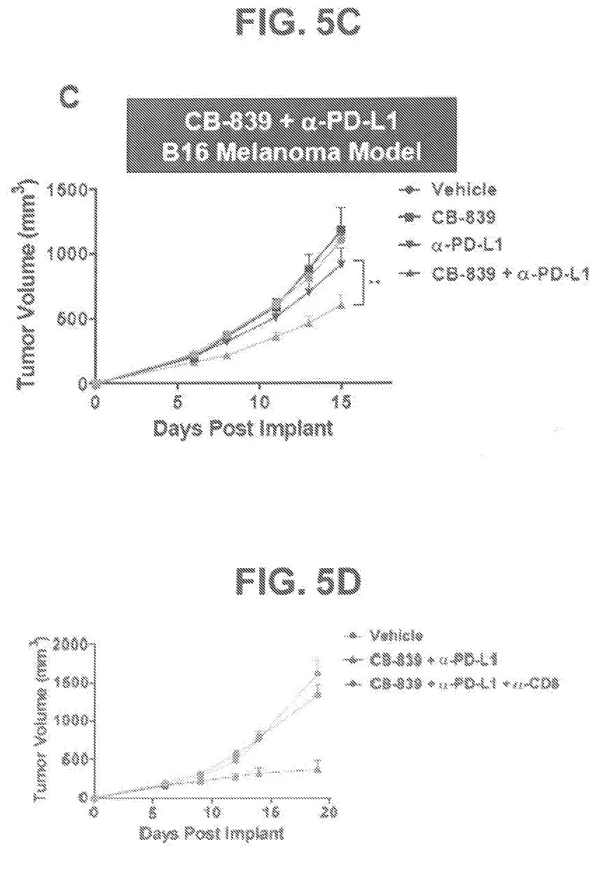

Balb/c mice were implanted subcutaneously with 1×10⁶ CT-26 colon carcinoma cells. Starting 24 hrs post implant, groups of 10 mice were treated with (i) vehicle dosed orally BID, (ii) CB-839 at 200 mg/kg dosed orally BID, (iii) α-PD-L1 (clone 10F.9G2, BioXCell) at 5 mg/kg dosed IP on days 5, 7, 9, 11, 13, and 15 and (iv) CB-839 and α-PD-L1. Tumor volumes are shown in FIG. 5A as an average and for individual animals. A synergistic effect of reducing tumor volumes was observed in mice receiving both CB-839 and α-PD-L1, such that 8 of 10 mice had a complete tumor regression.

The CT26 syngeneic model was used as above, except that α-PD-1 (clone RMP1-14, BioXCell) was used instead of α-PD-L1; α-PD-1 was dosed IP at 5 mg/kg on Days 6, 10 and 14. Tumor volumes are shown in FIG. 5B as an average and for individual animals. A synergistic effect of reducing tumor volumes was observed in mice receiving both CB-839 and α-PD-1.

C₅₇.B1/6 mice were implanted subcutaneously with 1×10⁶ B16 melanoma cells. Starting 24 hrs post implant, groups of 10 mice were treated with (i) vehicle dosed orally BID, (ii) CB-839 at 200 mg/kg orally BID, (iii) α-PD-L1 at 5 mg/kg dosed orally on days 6, 10 and 14, and (iv) CB-839 and α-PD-1. Tumor volumes are shown in FIG. 5C as an average and for individual animals. A synergistic effect of reducing tumor volumes was observed in mice receiving both CB-839 and α-PD-L1.

The CT26 model was used as in panel A, except that CD8+ cells were depleted by pre-treatment with an anti-CD8 antibody in one group treated with the combination of CB-839+α-PD-L1. Tumor volumes are shown in FIG. 5D as an average and for individual animals. Mice receiving both CB-839 and α-PD-L1 showed greater tumor growth, illustrating that depletion of CD8+ cells reverses the activity of this conjoint treatment.

### Example 3: Combination of CB-839 with Nivolumab in Treating Melanoma (MEL), Non-Small Cell Lung Cancer (NSCLC), and Renal Cell Carcinoma (RCC)

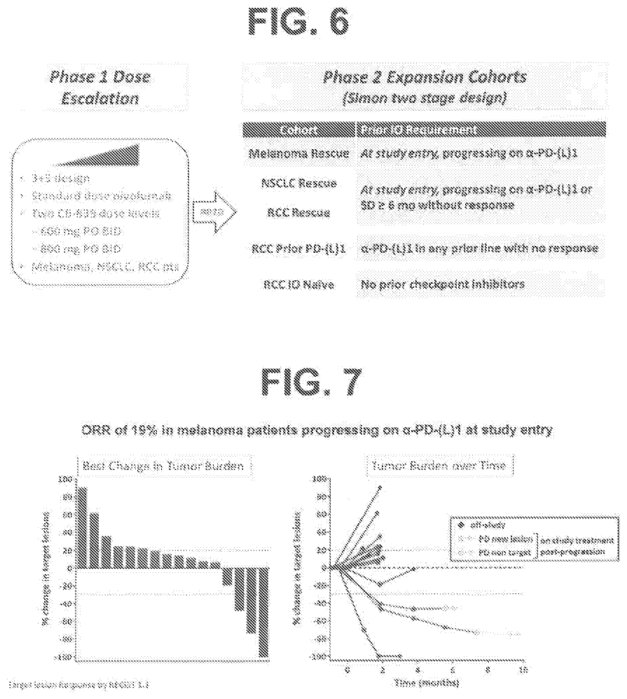

Human subjects having advanced/metastatic RCC, MEL, or NSCLC were treated with an escalating dose (or fixed dose) of CB-839 with a fixed dose of nivolumab. As shown in FIG. 6, the subjects received an anti-PD-1 therapy (e.g., nivolumab or pembrolizumab) as their most recent anti-cancer treatment with progression of disease (melanoma), or either progression of disease or stable disease without response for >6 months (NSCLC and RCC) and may have received multiple prior lines of IO therapy. The disease history qualified these patients as refractory to anti-PD-1 therapy.

In this study, the subjects received the FDA-approved dose of nivolumab (240 mg IV) on days 1 and 15 and a given dose of CB-839 orally twice daily on cycles lasting 28 days. Response to the treatment was evaluated using RECIST v1.1. The Kaplan-Meier method was used to estimate Progression-Free Survival (PFS), and overall survival (OS).

FIG. 7 shows the progress of the melanoma rescue patients, where an overall response rate (ORR) was 19% at the study entry and post-treatment, 10 patients had shown a partial to nearly complete response. The tumor burden decrease over time illustrates the efficacy of the present treatment in achieving beneficial responses in patients.

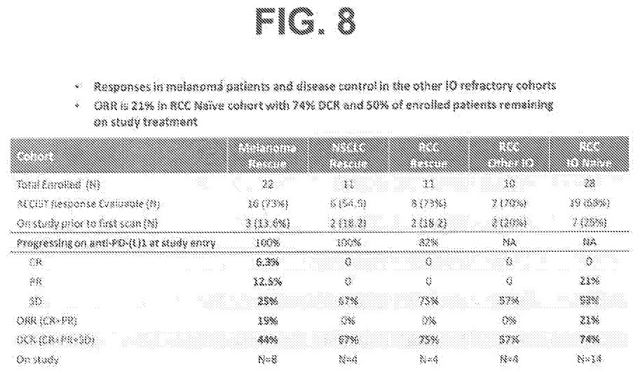

FIG. 8 provides data on all cohorts, showing that some melanoma rescue patients saw a complete (6.3%) or partial (12.5%) response to treatment while 25% of patients had stable disease. 44% of melanoma patients showed a positive outcome (DCR). In NSCLC rescue patients, 67% saw stable disease while in RCC rescue patients, 75% achieved stable disease. These results demonstrate the effectiveness of this treatment across cancer types.

### Example 4: Biomarker Study

Biopsies of patients undergoing treatment according to Example 3 were tested for gene expression of the biomarkers listed in Table 2. These patients had tumors that were inflamed. The biomarker expression was determined using the methods described in Ayers, et al. J Clin. Invest. 2017; 127(8):2930-2940. Ayers described a series of statistical quantile and housekeeping normalization analyses to determine cross-validated penalized regression models for the biomarker genes.

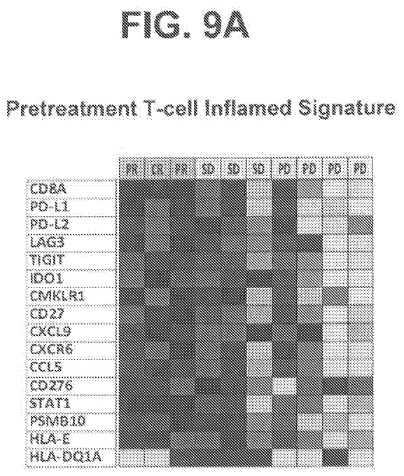

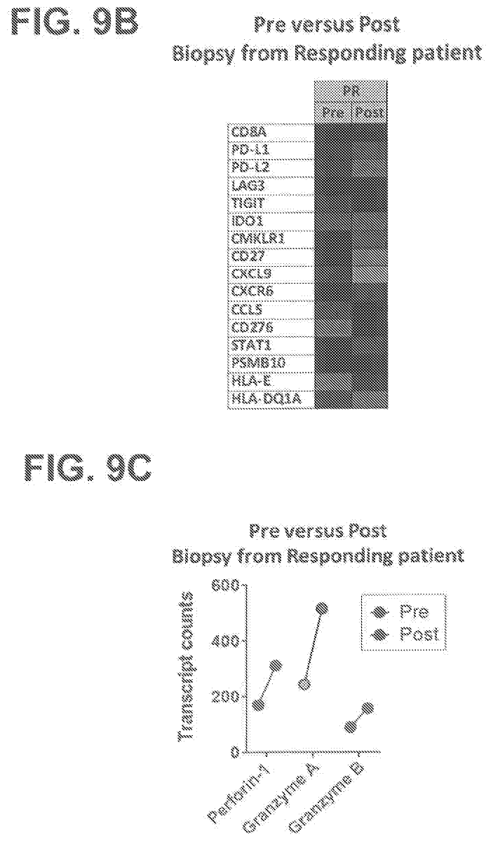

FIG. 9A shows that elevated levels of biomarkers related to T-cell inflamed signature in pretreatment biopsies associated with clinical benefit. Gene expression was analyzed in biopsies from Melanoma Rescue cohort. To further facilitate the visualization, transcript counts were replaced with colors. Low values are colored in green, high values are colored in red, and average values are colored in black. PR, partial response; CR, complete response; SD, stable disease; and PD, progressive disease.

In FIG. 9B, one patient with PR was evaluated for biomarker expression before and after treatment with CB-839 and nivolumab as described in Example 3. Elevation of nearly all biomarkers related to T-cell inflamed signature and effector genes were observed post-treatment with CB-839 and nivolumab.

In FIG. 9C, the PR patient assessed in FIG. 9B was evaluated for transcription (a sign of biomarker gene expression) of Perforin-1, Granzyme A and Granzyme B. All three of these biomarkers were elevated after treatment with CB-839 and nivolumab.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

In particular, suitable glutaminase inhibitors for practicing the invention are described in U.S. Pat. No. 8,604,016, U.S. application Ser. No. 14/081,175, and U.S. application Ser. No. 14/095,299, which are hereby incorporated by reference herein in their entirety.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating cancer in a subject refractory to treatment with a PD-1 or PD-L1 inhibitor, comprising conjointly administering to the subject a glutaminase inhibitor and a PD-1 or a PD-L1 inhibitor.

2. The method of claim 1, wherein the glutaminase inhibitor is a compound of formula (I), 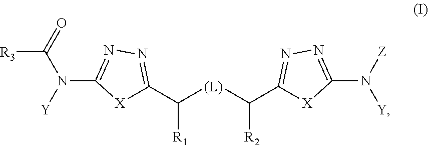
or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or 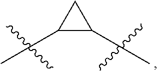 [SMILES: *C1CC1*]
preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂, CH₂CH₂CH₂ or CH₂ may be replaced by hydroxy;
X, independently for each occurrence, represents S, O or CH═CH, preferably S or CH═CH, wherein any hydrogen atom of a CH unit may be replaced by alkyl;
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy;
R₃, independently for each occurrence, represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₆, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H.

3. The method of claim 2, wherein L represents CH₂SCH₂, CH₂CH₂, CH₂S or SCH₂.

4. The method of claim 3, wherein L represents CH₂CH₂.

5. The method of any preceding claim, wherein Y represents H.

6. The method of any preceding claim, wherein X, independently for each occurrence, represents S or CH═CH, wherein any hydrogen atom of a CH unit may be replaced by alkyl.

7. The method of any preceding claim, wherein Z represents R₃(CO).

8. The method of claim 7, wherein each occurrence of R₃ is not identical.

9. The method of any preceding claim, wherein R₁ and R₂ each represent H.

10. The method of any preceding claim, wherein R₃, independently for each occurrence, represents substituted or unsubstituted arylalkyl, heteroarylalkyl, cycloalkyl or heterocycloalkyl.

11. The method of any one of claims 1-10, wherein R₃, independently for each occurrence, represents C(R₈)(R₉)(R₁₀), wherein R₈ represents substituted or unsubstituted aryl, arylalkyl, heteroaryl or heteroaralkyl, R₉ represents H, and R₁₀ represents hydroxy, hydroxyalkyl, alkoxy or alkoxyalkyl.

12. The method of claim 11, wherein R₈ represents substituted or unsubstituted aryl, arylalkyl, or heteroaryl.

13. The method of claim 11 or 12, wherein R₁₀ represents hydroxy, hydroxyalkyl, or alkoxy.

14. The method of claim 2, wherein L represents CH₂SCH₂, CH₂CH₂, CH₂S or SCH₂, Y represents H, X represents S, Z represents R₃(CO), R₁ and R₂ each represent H, and R₃, independently for each occurrence, represents substituted or unsubstituted arylalkyl, heteroarylalkyl, cycloalkyl or heterocycloalkyl.

15. The method of claim 14, wherein each occurrence of R₃ is identical.

16. The method of claim 2, wherein L represents CH₂SCH₂, CH₂CH₂, CH₂S or SCH₂, Y represents H, X represents S, Z represents R₃(CO), R₁ and R₂ each represent H, and R₃, independently for each occurrence, represents C(R₈)(R₉)(R₁₀), wherein R₈ represents substituted or unsubstituted aryl, arylalkyl, heteroaryl or heteroaralkyl, R₉ represents H, and R₁₀ represents hydroxy, hydroxyalkyl, alkoxy or alkoxyalkyl.

17. The method of claim 16, wherein L represents CH₂CH₂.

18. The method of claim 16 or 17, wherein R₈ represents substituted or unsubstituted aryl, arylalkyl or heteroaryl.

19. The method of claim 18, wherein R₈ represents substituted or unsubstituted aryl.

20. The method of any of claims 16-19, wherein R₁₀ represents hydroxy, hydroxyalkyl or alkoxy.

21. The method of claim 20, wherein R₁₀ represents hydroxyalkyl.

22. The method of any one of claims 16-21, wherein each occurrence of R₃ is identical.

23. The method of claim 2, wherein L represents CH₂CH₂, Y represents H, X, independently for each occurrence, represents S or CH═CH, Z represents R₃(CO), R₁ and R₂ each represent H, and R₃, independently for each occurrence, represents arylalkyl, heteroarylalkyl, cycloalkyl or heterocycloalkyl.

24. The method of claim 23, wherein each occurrence of R₃ is identical.

25. The method of any preceding claim, wherein the glutaminase inhibitor is a compound of formula (Ia), 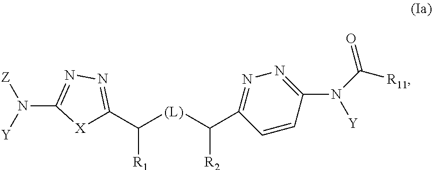
or a pharmaceutically acceptable salt thereof, wherein:
L represents CH₂SCH₂, CH₂CH₂, CH₂CH₂CH₂, CH₂, CH₂S, SCH₂, CH₂NHCH₂, CH═CH, or 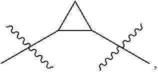 [SMILES: *C1CC1*]
preferably CH₂CH₂, wherein any hydrogen atom of a CH or CH₂ unit may be replaced by alkyl or alkoxy, any hydrogen of an NH unit may be replaced by alkyl, and any hydrogen atom of a CH₂ unit of CH₂CH₂, CH₂CH₂CH₂ or CH₂ may be replaced by hydroxy;
X represents S, O or CH═CH, preferably S or CH═CH, wherein any hydrogen atom of a CH unit may be replaced by alkyl;
Y, independently for each occurrence, represents H or CH₂O(CO)R₇;
R₇, independently for each occurrence, represents H or substituted or unsubstituted alkyl, alkoxy, aminoalkyl, alkylaminoalkyl, heterocyclylalkyl, arylalkyl, or heterocyclylalkoxy;
Z represents H or R₃(CO);
R₁ and R₂ each independently represent H, alkyl, alkoxy or hydroxy, preferably H;
R₃ represents substituted or unsubstituted alkyl, hydroxyalkyl, aminoalkyl, acylaminoalkyl, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, heteroaryloxyalkyl or C(R₈)(R₉)(R₁₀), N(R₄)(R₅) or OR₆, wherein any free hydroxyl group may be acylated to form C(O)R₇;
R₄ and R₅ each independently represent H or substituted or unsubstituted alkyl, hydroxyalkyl, acyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇;
R₆, independently for each occurrence, represents substituted or unsubstituted alkyl, hydroxyalkyl, aminoalkyl, acylaminoalkyl, alkenyl, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇; and
R₈, R₉ and R₁₀ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, or R₈ and R₉ together with the carbon to which they are attached, form a carbocyclic or heterocyclic ring system, wherein any free hydroxyl group may be acylated to form C(O)R₇, and wherein at least two of R₈, R₉ and R₁₀ are not H;
R₁₁ represents substituted or unsubstituted aryl, arylalkyl, aryloxy, aryloxyalkyl, heteroaryl, heteroarylalkyl, heteroaryloxy, or heteroaryloxyalkyl, or C(R₁₂)(R₃)(R₄), N(R₄)(R₁₄) or OR₁₄, wherein any free hydroxyl group may be acylated to form C(O)R₇;
R₁₂ and R₁₃ each independently represent H or substituted or unsubstituted alkyl, hydroxy, hydroxyalkyl, amino, acylamino, aminoalkyl, acylaminoalkyl, alkoxycarbonyl, alkoxycarbonylamino, alkenyl, alkoxy, alkoxyalkyl, aryl, arylalkyl, aryloxy, aryloxyalkyl, cycloalkyl, cycloalkylalkyl, heterocyclyl, heterocyclylalkyl, heteroaryl, heteroarylalkyl, heteroaryloxy, or heteroaryloxyalkyl, wherein any free hydroxyl group may be acylated to form C(O)R₇, and wherein both of R₁₂ and R₁₃ are not H; and
R₁₄ represents substituted or unsubstituted aryl, arylalkyl, aryloxy, aryloxyalkyl, heteroaryl, heteroarylalkyl, heteroaryloxy, or heteroaryloxyalkyl.

26. The method of claim 25, wherein Rn represents substituted or unsubstituted arylalkyl.

27. The method of claim 26, wherein R₁ represents substituted or unsubstituted benzyl.

28. The method of claim any of claims 25-27, wherein L represents CH₂SCH₂, CH₂CH₂, CH₂S or SCH₂.

29. The method of claim 28, wherein L represents CH₂CH₂.

30. The method of any of claims 25-29, wherein each Y represents H.

31. The method of any of claims 25-30, wherein X represents S or CH═CH.

32. The method of claim 31, wherein X represents S.

33. The method of any of claims 25-32, wherein Z represents R₃(CO).

34. The method of claim 33, wherein R₃ and R₁₁ are not identical.

35. The method of any of claims 25-34, wherein R₁ and R₂ each represent H.

36. The method of claim 33, wherein R₃ represents substituted or unsubstituted arylalkyl, heteroarylalkyl, cycloalkyl or heterocycloalkyl.

37. The method of claim 36, wherein R₃ represents substituted or unsubstituted heteroarylalkyl.

38. The method of claim 33, wherein R₃ represents C(R₈)(R₉)(R₁₀), wherein R₈ represents substituted or unsubstituted aryl, arylalkyl, heteroaryl or heteroaralkyl, R₉ represents H, and R₁₀ represents hydroxy, hydroxyalkyl, alkoxy or alkoxyalkyl.

39. The method of claim 38, wherein R₈ represents substituted or unsubstituted aryl, arylalkyl, or heteroaryl.

40. The method of claim 38 or 39, wherein R₁₀ represents hydroxy, hydroxyalkyl, or alkoxy.

41. The method of claim 25, wherein L represents CH₂SCH₂, CH₂CH₂, CH₂S or SCH₂, Y represents H, X represents S, Z represents R₃(CO), R₁ and R₂ each represent H, R₃ represents substituted or unsubstituted arylalkyl, heteroarylalkyl, cycloalkyl or heterocycloalkyl, and R₁ represents substituted or unsubstituted arylalkyl.

42. The method of claim 41, wherein R₃ represents substituted or unsubstituted heteroarylalkyl.

43. The method of claim 25, wherein L represents CH₂SCH₂, CH₂CH₂, CH₂S or SCH₂, Y represents H, X represents S, Z represents R₃(CO), R₁ and R₂ each represent H, R₃ represents C(R₈)(R₉)(R₁₀), wherein R₈ represents substituted or unsubstituted aryl, arylalkyl, heteroaryl or heteroaralkyl, R₉ represents H, R₁₀ represents hydroxy, hydroxyalkyl, alkoxy or alkoxyalkyl, and R₁₁ represents substituted or unsubstituted arylalkyl.

44. The method of claim 43, wherein R₈ represents substituted or unsubstituted aryl, arylalkyl or heteroaryl.

45. The method of claim 44, wherein R₈ represents heteroaryl.

46. The method of any of claims 43-45, wherein R₁₀ represents hydroxy, hydroxyalkyl or alkoxy.

47. The method of claim 25, wherein L represents CH₂CH₂, Y represents H, X represents S or CH═CH, Z represents R₃(CO), R₁ and R₂ each represent H, R₃ represents substituted or unsubstituted arylalkyl, heteroarylalkyl, cycloalkyl or heterocycloalkyl, and R₁₁ represents substituted or unsubstituted arylalkyl.

48. The method of claim 47, wherein R₃ represents substituted or unsubstituted heteroarylalkyl.

49. The method of claim 25, wherein L represents CH₂CH₂, Y represents H, X represents S, Z represents R₃(CO), R₁ and R₂ each represent H, R₃ represents C(R₈)(R₉)(R₁₀), wherein R₈ represents substituted or unsubstituted aryl, arylalkyl or heteroaryl, R₉ represents H, R₁₀ represents hydroxy, hydroxyalkyl or alkoxy, and R₁₁ represents substituted or unsubstituted arylalkyl.

50. The method of claim 25, wherein R₈ represents H, R₉ represents H, and R₁₀ represents heteroaryl.

51. The method of claim 1, wherein the glutaminase inhibitor is 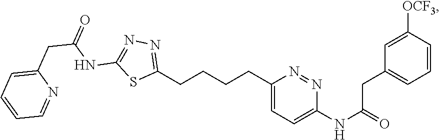 [SMILES: O=C(Cc1cccc(OC(F)(F)F)c1)Nc1ccc(CCCCc2nnc(NC(=O)Cc3ccccn3)s2)nn1]
or a pharmaceutically acceptable salt thereof.

52. The method of any preceding claim, wherein the cancer is refractory to a PD-1 or PD-L1 inhibitor selected from bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head & neck cancer, Kaposi's sarcoma, kidney cancer, leukemia, liver cancer, lung cancer, lymphoma, melanoma, myeloma, ovarian cancer, pancreatic cancer, penile cancer, prostate cancer, renal cell cancer, testicular germ cell cancer, thymoma and thymic carcinoma.

53. The method of any preceding claim, wherein the cancer is melanoma.

54. The method of any preceding claim, wherein the cancer is non-small cell lung cancer.

55. The method of any preceding claim, wherein the cancer is renal cell carcinoma.

56. The method of any preceeding claim, wherein the PD-1 or the PD-L1 inhibitor is an anti-PD-1 or an anti-PD-L1 antibody.

57. The method of claim 56, wherein the PD-1 or the PD-L1 inhibitor is a anti-PD-1 or an anti-PD-L1 antibody selected from nivolumab, pembrolizumab, pidilizumab, ipilimumab, atezolizumab, avelumab and durvalumab.

58. The method of any preceding claim, wherein the glutaminase inhibitor and the PD-1 or the PD-L1 inhibitor are administered simultaneously.

59. The method of any one of claims 1-58, wherein the glutaminase inhibitor is administered within about 5 minutes to within about 168 hours prior to or after administration of the PD-1 or PD-L1 inhibitor.

60. The method of any preceding claim, wherein the PD-1 or PD-L1 inhibitor administered to the subject is the PD-1 or PD-L1 inhibitor to which the subject is refractory.

61. The method of any preceding claim, further comprising conjointly administering one or more additional chemotherapeutic agents.

62. The method of claim 61, wherein the one or more additional chemotherapeutic agents are selected from ABT-263, afatinib dimaleate, axitinib, aminoglutethimide, amsacrine, anastrozole, asparaginase, AZD5363, Bacillus Calmette-Guerin vaccine (bcg), bicalutamide, bleomycin, bortezomib, buserelin, busulfan, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, procarbazine, raltitrexed, ramucirumab, rituximab, romidepsin, rucaparib, selumetinib, sirolimus, sorafenib, streptozocin, sunitinib, suramin, talazoparib, tamoxifen, temozolomide, temsirolimus, teniposide, testosterone, thalidomide, thioguanine, thiotepa, titanocene dichloride, topotecan, trametinib, trastuzumab, tretinoin, veliparib, vinblastine, vincristine, vindesine, vinorelbine, and vorinostat (SAHA).

63. The method of claim 61, wherein the one or more additional chemotherapeutic agents are selected from bortezomib, capecitabine, carboplatin, carfilzomib, cyclophosphamide, daunorubicin, doxorubicin, epirubicin, eribulin, fluorouracil, gemcitabine, ixabepilone, lenalidomide, methotrexate, mitoxantrone, mutamycin, rituximab, thiotepa, vincristine, and vinorelbine.

64. The method of claim 61, wherein the one or more additional chemotherapeutic agents are selected from bortezomib, carfilzomib, doxorubicin, lenalidomide, and rituximab.

65. The method of claim 61, wherein the additional chemotherapeutic agent is an immuno-oncology agent.

66. The method of claim 65, wherein the immuno-oncology agent is an anti-CTLA-4 agent selected from ipilimumab and tremelimumab.

67. A method for identifying the likelihood of a cancer selected from melanoma, non-small cell lung cancer (NSCLC), and renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:
a) obtaining or providing a tumor sample from a subject having the cancer;
b) measuring the presence, absence, amount, or activity of at least one biomarker listed in Table 2 in the tumor sample; and
c) comparing said presence, absence, amount, or activity of the at least one biomarker listed in Table 2 to a reference standard representative of a non-responsive refractory tumor,
wherein the presence of the at least one biomarker listed in Table 2 or a significantly increased amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

68. A method for identifying the likelihood of a cancer selected from melanoma, non-small cell lung cancer (NSCLC), and renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:
a) obtaining or providing a sample from a subject having the cancer, wherein the sample comprises nucleic acid molecules from the tumor;
b) determining the copy number of at least one biomarker listed in Table 2 in the sample; and
c) comparing the copy number to a reference standard representative of a non-responsive refractory tumor,
wherein an increased copy number of the at least one biomarker listed in Table 2 in the sample relative to the reference standard identifies the cancer as being more likely to be responsive to the conjoint therapy.

69. A method of monitoring an effect of conjoint therapy with a glutaminase inhibitor and a PD-1 or a PD-L1 inhibitor to treat a cancer selected from melanoma, non-small cell lung cancer (NSCLC), and renal cell carcinoma (RCC), in a subject refractory to treatment with a PD-1 or PD-L1 inhibitor, comprising:
(a) obtaining a tumor sample from the subject; and
(b) evaluating expression in the sample of at least one biomarker listed in Table 2, or a combination thereof, compared to a reference standard representative of a non-responsive refractory tumor,
wherein an increased expression of the at least one biomarker, or a combination thereof, relative to the reference standard, indicates that the conjoint therapy is effective.

70. A method of monitoring an effect of conjoint therapy with a glutaminase inhibitor and a PD-1 or a PD-L1 inhibitor to treat a cancer selected from melanoma, non-small cell lung cancer (NSCLC), and renal cell carcinoma (RCC), in a subject refractory to treatment with a PD-1 or PD-L1 inhibitor, comprising:
(a) obtaining a tumor sample from the subject; and
(b) determining the copy number of at least one biomarker listed in Table 2 in the sample; and
c) comparing the copy number to a reference standard representative of a non-responsive refractory tumor,
wherein an increased copy number of the at least one biomarker listed in Table 2 in the sample relative to the reference standard indicates that the conjoint therapy is effective.

71. A method for identifying the likelihood of a cancer selected from melanoma, non-small cell lung cancer (NSCLC), and renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:
a) obtaining or providing a tumor sample from a subject having the cancer;
b) measuring the presence, absence, amount, or activity of at least one biomarker listed in Table 2 in the tumor sample; and
c) comparing said presence, absence, amount, or activity of the at least one biomarker listed in Table 2 to a reference standard representative of a responsive refractory tumor,
wherein the presence of the at least one biomarker listed in Table 2 or a similar amount or activity of the at least one biomarker listed in Table 2, in the tumor sample relative to the reference standard identifies the cancer as being more likely to be responsive to conjoint therapy with the glutaminase inhibitor and a PD-1 or a PD-L1 inhibitor.

72. A method of identifying the likelihood of a cancer selected from melanoma, non-small cell lung cancer (NSCLC), and renal cell carcinoma (RCC), in a subject refractory to treatment with a PD-1 or PD-L1 inhibitor to be responsive to conjoint therapy with a glutaminase inhibitor and a PD-1 or a PD-L1 inhibitor, the method comprising:
a) obtaining or providing a sample from a subject having the cancer, wherein the sample comprises nucleic acid molecules from the tumor;
b) determining the copy number of at least one biomarker listed in Table 2 in the sample; and
c) comparing the copy number to a reference standard representative of a responsive refractory tumor,
wherein a similar copy number of the at least one biomarker listed in Table 2 in the sample relative to the reference standard identifies the cancer as being more likely to be responsive to the conjoint therapy.

73. A method of monitoring an effect of conjoint therapy with a glutaminase inhibitor and a PD-1 or a PD-L1 inhibitor to treat a cancer selected from melanoma, non-small cell lung cancer (NSCLC), and renal cell carcinoma (RCC), in a subject refractory to treatment with a PD-1 or PD-L1 inhibitor, comprising:
(a) obtaining a tumor sample from the subject; and
(b) evaluating expression in the sample of at least one biomarker listed in Table 2, or a combination thereof, compared to a reference standard representative of a responsive refractory tumor,
wherein a similar expression of the at least one biomarker, or a combination thereof, relative to the reference standard, indicates that the conjoint therapy is effective.

74. A method of monitoring an effect of conjoint therapy with a glutaminase inhibitor and a PD-1 or a PD-L1 inhibitor to treat a cancer selected from melanoma, non-small cell lung cancer (NSCLC), and renal cell carcinoma (RCC), in a subject refractory to treatment with a PD-1 or PD-L1 inhibitor, comprising:
(a) obtaining a tumor sample from the subject; and
(b) determining the copy number of at least one biomarker listed in Table 2 in the sample; and
c) comparing the copy number to a reference standard representative of a responsive refractory tumor,
wherein a similar copy number of the at least one biomarker listed in Table 2 in the sample relative to the reference standard indicates that the conjoint therapy is effective.

75. The method of any one of claims 67, 68, 71, and 72, wherein
the cancer is identified to be likely to be responsive to the conjoint therapy; and
the method further comprises administering the conjoint therapy to the subject.

76. The method of any one of claims 69, 70, 73, and 74 wherein
the conjoint therapy is identified to be effective; and
the method further comprises continuing to administer the conjoint therapy to the subject.

77. The method of any one of claims 67-76, wherein the reference standard comprises cancer cells known to be responsive or non-responsive to the glutaminase inhibitor and a PD1 or a PD-L1 inhibitor conjoint therapy.

78. The method of claim 67 or 71, wherein the presence or amount of the at least one biomarker listed in Table 2 is detected using a reagent which specifically binds with the protein and is selected from an antibody, an antibody derivative, and an antibody fragment.

79. The method of claim 67 or 71, wherein the presence or amount of the at least one biomarker listed in Table 2 is assessed by detecting the presence in the sample of a transcribed polynucleotide or portion thereof.

80. The method of any one of claims 67-79, wherein the cancer is melanoma.

81. The method of any one of claims 67-80, wherein the cancer is non-small cell lung cancer.

82. The method of any one of claims 67-81, wherein the cancer is renal cell cancer.
